(19) United States
(12) Patent Application Publication
(10) Pub. No.: US 2013/0018018 A1
(43) Pub. Date: Jan. 17, 2013

(54) NOVEL NUCLEOSIDE PHOSPHONATES AND ANALOGS

(71) Applicants: Steven Dong, San Francisco, CA (US); Peter G.M. Wuts, Kalamazoo, MI (US)

(72) Inventors: Steven Dong, San Francisco, CA (US); Peter G.M. Wuts, Kalamazoo, MI (US)

(73) Assignee: Epiphany Biosciences ,Inc., San Francisco, CA (US)

(21) Appl. No.: 13/391,145

(22) PCT Filed: Aug. 27, 2010
(86) PCT No.: PCT/US2010/047054

§ 371 Date: Oct. 2, 2012

Related U.S. Application Data
(60) Provisional application No. 61/237,679, filed on Aug. 27, 2009.

(51) Int. Cl. C07F 9/6561; A61P 31/12; A61K 31/675

(52) U.S. Cl. 514/81; 544/244

(57) ABSTRACT

Compounds and compositions are provided for treatment, prevention, or amelioration of a variety of medical disorders associated with viral infections and/or cell proliferation. The compounds provided herein are nucleoside phosphonates and esters thereof.

### FIELD

Provided herein are nucleoside phosphonates and esters thereof. In some embodiments, the compounds are monoesters of biologically active nucleosides and analogs thereof. In other embodiments, provided herein are methods of treatment, prevention, or amelioration of a variety of medical disorders associated with viral infections and cell proliferation using the compounds and compositions provided herein.

### BACKGROUND

Nucleoside phosphonates have long been known to have antiviral, antiproliferative and a variety of other therapeutic benefits. Among these are the antiviral nucleoside phosphonates, such as, for example, cidofovir, cyclic cidofovir, adefovir, tenofovir, and the like, as well as the 5′-phosphonates and methylene phosphonates of azidothymidine (AZT), ganciclovir, acyclovir, and the like. In these compounds, the 5′-hydroxyl of the sugar moiety, or its equivalent in acyclic nucleosides (ganciclovir, penciclovir, acyclovir) which do not contain a complete sugar moiety, is replaced with a phosphorus-carbon bond. In the case of the methylene phosphonates, a methylene group replaces the 5′-hydroxyl or its equivalent, and its carbon atom is, in turn, covalently linked to the phosphonate.

Such compounds may be active as antiviral or antiproliferative nucleotides. Upon cellular metabolism, two additional phosphorylations occur to form the nucleoside phosphonate diphosphate which represents the equivalent of nucleoside triphosphates. Antiviral nucleoside phosphonate diphosphates are selective inhibitors of viral RNA or DNA polymerases or reverse transcriptases. Accordingly, their inhibitory action on viral polymerases is much greater than their degree of inhibition of mammalian cell DNA polymerases alpha, beta and gamma or mammalian RNA polymerases. Conversely, the antiproliferative nucleoside phosphonate diphosphates inhibit cancer cell DNA and RNA polymerases and may show much lower selectivity versus normal cellular DNA and RNA polymerases.

There is a continuing need for less toxic, more effective pharmaceutical agents to treat a variety of disorders associated with viral infection, and cell proliferation.

### SUMMARY

Provided herein are nucleoside phosphonates and lipophilic esters thereof. Also provided are compositions and methods of using the compounds and compositions for the treatment of various diseases. In some embodiments, compounds and compositions provided herein have antiviral activity. In other embodiments, provided herein are compounds and compositions that are useful in the treatment, prevention, or amelioration of one or more symptoms associated with cell proliferation.

In certain embodiments, the compounds are nucleoside phosphonates and pharmaceutically acceptable derivatives thereof. In other embodiments, the compounds are lipophilic esters of nucleoside phosphonates.

In some embodiments, compounds of Formula (I) or Formula (II):

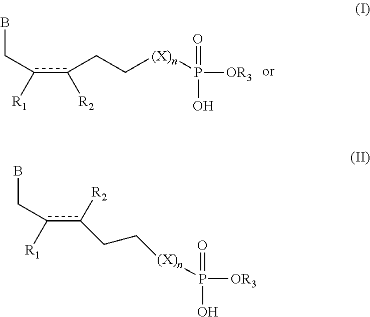

or salts, solvated or hydrates thereof are provided wherein:
B is a purine or pyrimidine base or an analog thereof;
R₁ and R₂ are independently hydrogen, alkyl, substituted alkyl, aryl, substituted aryl, arylalkyl, substituted arylalkyl, R₄CO₂—, R₅O—, —R₆S(O)ₖ, halo, heteroalkyl, —N₃, substituted heteroalkyl, heteroaryl, substituted heteroaryl, heteroarylalkyl or substituted heteroarylalkyl or alternatively, in the compound of Formula (I), R₁ and R₂ together with the carbon atoms to which they are bonded form a cycloalkyl, substituted cycloalkyl, cycloheteroalkyl or substituted cycloheteroalkyl ring;

### X is —CH₂—;

n is 0 or 1;
k is 0, 1 or 2;
R₃ is hydrogen, a monovalent cation or a lipophilic group; and
R₄, R₅, and R₆ are independently alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl;

provided that at least one of R₁ and R₂ is not hydrogen or that when both R₁ and R₂ are hydrogen, B is

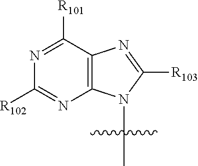

wherein R₁₀₁ is −OR₁₀₄, —SR₁₀₅, —NR₁₀₆NH₂ or —NR₁₀₇NHSO₂Me, R₁₀₂ is hydrogen, alkyl, halo or —NR₁₀₈R₁₀₉, R₁₀₃ is hydrogen or alkyl and R₁₀₄, R₁₀₅, R₁₀₆, R₁₀₇, R₁₀₈ and R₁₀₉ are independently hydrogen or alkyl;

provided that when R₁ and R₂ together with the atoms to which they are bonded form a cycloalkyl, substituted cycloalkyl, cycloheteroalkyl or substituted cycloheteroalkyl ring a double bond is optionally present between the carbon atoms connecting R₁ and R₂; and

provided that when R₁ and R₂ are independently hydrogen, alkyl, substituted alkyl, aryl, substituted aryl, arylalkyl, substituted arylalkyl, R₄CO₂—, R₅O—, —R₆S(O)ₖ, halo, heteroalkyl, —N₃, substituted heteroalkyl, heteroaryl, substituted heteroaryl, heteroarylalkyl or substituted heteroarylalkyl a double bond is optionally present between the carbon atoms connecting R₁ and R₂.

In other aspects, compounds of Formula (III):

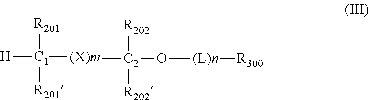

or salts, solvated or hydrated thereof are provided wherein:
R₂₀₁ and R_{201′}, are independently hydrogen, —OR₂₀₃, —OC(O)_{R204}, —SR₂₀₅ or —SC(O)R₂₀₆, R₂₀₃, R₂₀₄, R₂₀₅ or R₂₀₆ are independently alkyl, R₂₀₂ and R_{202′}, are independently hydrogen, —OR₂₀₇, —OC(O)R₂₀₈, —SR₂₀₉, —SC(O)R₂₁₀, —NR₂₁₁R₂₁₂, or —NC(O)R₂₁₃, R₂₀₇, R₂₀₉, R₂₁₁ or R₂₁₂ are hydrogen or alkyl and R₂₀₈, R₂₁₀ and R₂₁₃ are alkyl;
R₃₀₀ is a phosphonate containing a nucleotide phosphonate or an antiviral phosphonate which substitutes the pyrimidine base R₃₀₁ for the purine or pyrimidine base of the nucleotide or nucleoside or the antiviral phosphonate

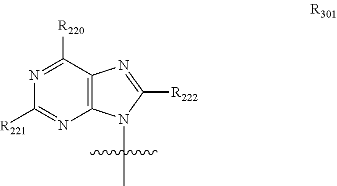

wherein R₂₂₀ is hydrogen, hydroxyl, halo, alkyl, —OR₂₂₄, —SR₂₂₅, —NR₂₂₆NH₂, —NR₂₂₇NHSO₂Me or —NR₂₂₈R₂₂₉, R₂₂₁ is hydrogen, alkyl, halo or —NR₂₂₈R₂₂₉, R₂₂₂ is hydrogen or alkyl and R₂₂₄, R₂₂₅, R₂₂₆NH₂, R₂₂₇, R₂₂₈ and R₂₂₉ are hydrogen or alkyl;

### X is —(R₂₀₂)C(R_{202′})—;

L is a valence bond or a bifunctional linking molecule of the formula -J-(CR₂₃₀)ₜ-G- wherein t is an integer from 1 to 24, J and G are independently —O—, —S—, —C(O)O— or —NH— and R₂₃₀ is hydrogen, alkyl or substituted alkyl;
m is an integer from 0 to 6;
n is 0 or 1;
provided that both R₂₀₁ and R_{201′}, are not OC(O)R₂₀₄, —SC(O)R₂₀₆, or combinations thereof; and provided that both R₂₀₂ and R_{202′}, attached to the same carbon atom are not both —OC(O)R₂₀₈, —SC(O)R₂₁₀, —NR₂₁₁R₂₁₂, or —NC(O)R₂₁₃.

Also provided are pharmaceutically acceptable derivatives, including salts, esters, enol ethers, enol esters, solvates, hydrates and prodrugs of the compounds described herein. Further provided are pharmaceutical compositions containing the compounds provided herein and a pharmaceutically acceptable vehicle. In some embodiments, the pharmaceutical compositions are formulated for single dosage administration.

Methods of treating, preventing, or ameliorating one or more symptoms of diseases associated with viral infections and cell proliferation using the compounds and compositions provided herein are provided. In practicing the methods, therapeutically effective amounts of the compounds or compositions containing therapeutically effective concentrations of the compounds are administered.

Articles of manufacture are provided containing packaging material, a compound or composition provided herein which is useful for treating, preventing, or ameliorating one or more symptoms of diseases or disorders associated with viral infections or cell proliferation using the compounds and compositions provided herein, and a label that indicates that the compound or composition is useful for treating, preventing, or ameliorating one or more symptoms of diseases or disorders associated with viral infections or cell proliferation.

### DETAILED DESCRIPTION

### Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

“Nucleoside base” refers to natural and non-natural purine and pyrimidine bases, including adenine, thymine, cytosine, guanine and uracil and analogs thereof. When the nucleoside base contains 1 or more functional groups that may be reactive to form undesired products under the reaction conditions used for preparing the compounds provided herein, for example, the amino groups of cytosine and adenine and the 2-amino and 6-oxo groups of guanine, such functional groups may be blocked using the protecting groups commonly employed in nucleoside chemistry. For example, the amino group of adenine and cytosine may be protected by benzoyl; the 6-oxo and 2-amino groups of guanine may be protected by the triphenylmethyl (trityl) group. The selection of methods for introducing and subsequent removal of such protecting groups are well known to one of ordinary skill in the pertinent art.

“Lipophilic” or “long-chain” refer to the cyclic, branched or straight chain chemical groups that when covalently linked to a phosphonic acid to form a phosphonate monoester, increase oral bioavailability and enhance activity of the nucleoside phosphonates as compared with the parent nucleoside phosphonates. These lipophilic groups include, but are not limited to alkyl, alkoxyalkyl, and alkylglyceryl. In some embodiments, the alkyl groups contain from 8-26 carbon atoms or 8, 10, 12, 13, 14, 15, 16, 17, 18, 19, 20, 21, 22, 23 or 24 carbon atoms and can be straight or branched chain moieties.

“Nucleoside phosphonate” and “acyclic nucleoside phosphonate” refer to the group of phosphonomethoxyalkyl or phosphono substituted nucleoside derivatives that are biologically active, for example, as antiviral, anti-cancer or anti-parasitic drugs.

“Lipophilic monoesters of nucleoside phosphonates” refers to a compound where a lipophilic group is covalently attached to a nucleoside phosphonate via an ester linkage.

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In some embodiments, an alkyl group comprises from 1 to 20 carbon atoms (C₁-C₂₀ alkyl). In other embodiments, an alkyl group comprises from 1 to 10 carbon atoms (C₁-C₁₀ alkyl). In still other embodiments, an alkyl group comprises from 1 to 6 carbon atoms (C₁-C₆ alkyl).

“Alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl,” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkoxy,” by itself or as part of another substituent, refers to a radical of the formula —O—R⁴⁰⁰, where R⁴⁰⁰ is alkyl or substituted alkyl as defined herein.

“Acyl” by itself or as part of another substituent refers to a radical —C(O)R⁴⁰¹, where R⁴⁰¹ is hydrogen, alkyl, substituted alkyl, aryl, substituted aryl, arylalkyl, substituted arylalkyl, heteroalkyl, substituted heteroalkyl, heteroarylalkyl or substituted heteroarylalkyl as defined herein. Representative examples include, but are not limited to formyl, acetyl, cyclohexylcarbonyl, cyclohexylmethylcarbonyl, benzoyl, benzylcarbonyl and the like.

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryl group comprises from 6 to 20 carbon atoms (C₆-C₂₀ aryl). In other embodiments, an aryl group comprises from 6 to 15 carbon atoms (C₆-C₁₅ aryl). In still other embodiments, an aryl group comprises from 6 to 15 carbon atoms (C₆-C₁₀ aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group, as defined herein. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In some embodiments, an arylalkyl group is (C₆-C₃₀) arylalkyl, e.g., the alkanyl, alkenyl or alkynyl moiety of the arylalkyl group is (C₁-C₁₀) alkyl and the aryl moiety is (C₆-C₂₀) aryl. In other embodiments, an arylalkyl group is (C₆-C₂₀) arylalkyl, e.g., the alkanyl, alkenyl or alkynyl moiety of the arylalkyl group is (C₁-C₈) alkyl and the aryl moiety is (C₆-C₁₂) aryl. In still other embodiments, an arylalkyl group is (C₆-C₁₅) arylalkyl, e.g., the alkanyl, alkenyl or alkynyl moiety of the arylalkyl group is (C₁-C₅) alkyl and the aryl moiety is (C₆-C₁₀) aryl.

“Cycloalkyl,” by itself or as part of another substituent, refers to a saturated or unsaturated cyclic alkyl radical, as defined herein. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In some embodiments, the cycloalkyl group comprises from 3 to 10 ring atoms (C₃-C₁₀ cycloalkyl). In other embodiments, the cycloalkyl group comprises from 3 to 7 ring atoms (C₃-C₇ cycloalkyl).

“Cycloheteroalkyl,” by itself or as part of another substituent, refers to a saturated or unsaturated cyclic alkyl radical in which one or more carbon atoms (and optionally any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidone, quinuclidine, and the like. In some embodiments, the cycloheteroalkyl group comprises from 3 to 10 ring atoms (3-10 membered cycloheteroalkyl). In other embodiments, the cycloalkyl group comprise from 5 to 7 ring atoms (5-7 membered cycloheteroalkyl). A cycloheteroalkyl group may be substituted at a heteroatom, for example, a nitrogen atom, with a (C₁-C₆) alkyl group. As specific examples, N-methyl-imidazolidinyl, N-methyl-morpholinyl, N-methyl-piperazinyl, N-methyl-piperidinyl, N-methyl-pyrazolidinyl and N-methyl-pyrrolidinyl are included within the definition of “cycloheteroalkyl.” A cycloheteroalkyl group may be attached to the remainder of the molecule via a ring carbon atom or a ring heteroatom.

“Compounds” refers to compounds encompassed by structural formulae disclosed herein and includes any specific compounds within these formulae whose structure is disclosed herein. Compounds may be identified either by their chemical structure and/or chemical name. When the chemical structure and chemical name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. In general, all physical forms are equivalent for the uses contemplated herein and are intended to be within the scope of the present invention. Further, it should be understood, when partial structures of the compounds are illustrated, that brackets indicate the point of attachment of the partial structure to the rest of the molecule. Also it should be understood that the denotion [symbol] in compounds of Formula (I) and (II) denote a double bond which is optionally present depending on other restrictions.

“Halo,” by itself or as part of another substituent refers to a radical —F, —Cl, —Br or —I.

“Heteroalkyl,” “Heteroalkanyl,” “Heteroalkenyl” and “Heteroalkynyl,” by themselves or as part of other substituents, refer to alkyl, alkanyl, alkenyl and alkynyl groups, respectively, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰²—, ═N—N═, —N═N—, —N═N—NR⁵⁰³R⁴⁰⁴, —PR⁵⁰⁵—, —P(O)₂—, —POR⁵⁰⁶—, —O—P(O)₂—, —SO—, —SO₂—, —SnR⁵⁰⁷R⁵⁰⁸— and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl,” by itself or as part of another substituent, refers to a monovalent heteroaromatic radical derived by the removal of one hydrogen atom from a single atom of a parent heteroaromatic ring systems, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group comprises from 5 to 20 ring atoms (5-20 membered heteroaryl). In other embodiments, the heteroaryl group comprises from 5 to 10 ring atoms (5-10 membered heteroaryl). Exemplary heteroaryl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heteroarylalkynyl is used. In some embodiments, the heteroarylalkyl group is a 6-21 membered heteroarylalkyl, e.g., the alkanyl, alkenyl or alkynyl moiety of the heteroarylalkyl is (C₁-C₆) alkyl and the heteroaryl moiety is a 5-15-membered heteroaryl. In other embodiments, the heteroarylalkyl is a 6-13 membered heteroarylalkyl, e.g., the alkanyl, alkenyl or alkynyl moiety is (C₁-C₃) alkyl and the heteroaryl moiety is a 5-10 membered heteroaryl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s are taken together with the nitrogen atom to which they are bonded form a 4-, 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl.

Similarly, substituent groups useful for substituting unsaturated carbon atoms in the specified group or radical include, but are not limited to, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups useful for substituting nitrogen atoms in heteroalkyl and cycloheteroalkyl groups include, but are not limited to, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂O, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

“Subject,” “individual” or “patient” are used interchangeably herein and refer to a vertebrate, preferably a mammal. Mammals include, but are not limited to, murines, rodents, simians, humans, farm animals, sport animals and pets.

“Treating” or “treatment” of any disease or disorder refers, in some embodiments, to ameliorating the disease or disorder (i.e., arresting or reducing the development of the disease or at least one of the clinical symptoms thereof). In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter) or both. In yet other embodiments, “treating” or “treatment” refers to delaying the onset of the disease or disorder.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, etc., of the patient to be treated.

“Vehicle” refers to a diluent, adjuvant, excipient or carrier with which a compound is administered.

The abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

Some abbreviations used herein are as follows:

5-Phosphono-pent-2-en-1-yl adenine=PPen-A,

5-Phosphono-pent-2-en-1-yl cytosine=PPen-C,

5-Phosphono-pent-2-en-1-yl guanine=PPen-G,

5-Phosphono-pent-2-en-1-yl thymine=PPen-T and

5-Phosphono-pent-2-en-1-yl uracil=PPen-U,

Hexadecyloxypropyl=HDP

Octadecyloxyethyl=ODE

Oleyloxyethyl=OLE, and

Oleyloxypropyl=OLP.

B. Compounds

In a first aspect, substituted phosphonates of Formula (I) or Formula (II):

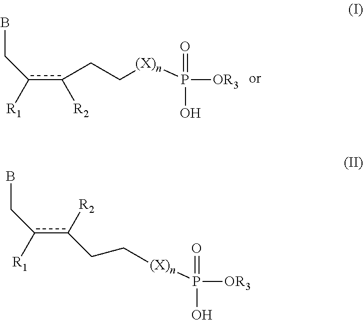

or salts, solvates or hydrates thereof are provided wherein B is a purine or pyrimidine base or an analog thereof, R₁ and R₂ are independently hydrogen, alkyl, substituted alkyl, aryl, substituted aryl, arylalkyl, substituted arylalkyl, R₄CO₂—, R₅O—, —R₆S(O)ₖ, halo, heteroalkyl, —N₃, substituted heteroalkyl, heteroaryl, substituted heteroaryl, heteroarylalkyl or substituted heteroarylalkyl or alternatively, in the compound of Formula (I), R₁ and R₂ together with the carbon atoms to which they are bonded form a cycloalkyl, substituted cycloalkyl, cycloheteroalkyl or substituted cycloheteroalkyl ring; X is —CH₂—, n is 0 or 1, k is 0, 1 or 2, R₃ is hydrogen, a monovalent cation or a lipophilic group and R₄, R₅, and R₆ are independently alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl, provided that at least one of R₁ and R₂ is not hydrogen or that when both R₁ and R₂ are hydrogen, B is

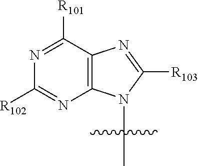

wherein R₁₀₁ is —OR₁₀₄, —SR₁₀₅, —NR₁₀₆NH₂ or —NR₁₀₇NHSO₂Me, R₁₀₂ is hydrogen, alkyl, halo or —NR₁₀₈R₁₀₆, R₁₀₃ is hydrogen or alkyl and R₁₀₄, R₁₀₅, R₁₀₆, R₁₀₇, R₁₀₈ and R₁₀₉ are independently hydrogen or alkyl provided that when R₁ and R₂ together with the atoms to which they are bonded form a cycloalkyl, substituted cycloalkyl, cycloheteroalkyl or substituted cycloheteroalkyl ring a double bond is optionally present between the carbon atoms connecting R₁ and R₂; and provided that when R₁ and R₂ are independently hydrogen, alkyl, substituted alkyl, aryl, substituted aryl, arylalkyl, substituted arylalkyl, R₄CO₂—, R₅O—, —R₆S(O)ₖ, halo, heteroalkyl, —N₃, substituted heteroalkyl, heteroaryl, substituted heteroaryl, heteroarylalkyl or substituted heteroarylalkyl a double bond is optionally present between the carbon atoms connecting R₁ and R₂.

In some embodiments, a compound of Formula (I) is provided where B, R₁, R₂, R₃, X and n are as defined above. In other embodiments, in the compound of Formula (II), R₃ is not hydrogen. In still other embodiments, in the compound of Formula (II), R₃ is not hydrogen when B is cytosine or uracil.

In some embodiments, R₃ is hydrogen, a monovalent cation, C₈-C₂₄ alkyl or substituted C₈-C₂₄ alkyl. In other embodiments, R₃ is substituted C₈-C₂₄ alkyl. In still other embodiments, R₃ is substituted with 1 to 3 halo, —OH, —SH, —O— or alkyl groups. In still other embodiments, R₃ is R₇CO— and R₇ is alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl. In still other embodiments, R₃ is acetyl, valyl, dipivoxil, bis(pivaloyloxymethyl) or disoproxil. In still other embodiments, R₃ is acetyl, valyl or dipivoxil.

In some embodiments, R₃ is:

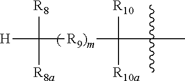

wherein R₈ and R₈ₐ are independently, hydrogen, R₁₁CO₂—, R₁₂O—, R₁₃S— or R₁₄COS— and R₁₁, R₁₂, R₁₃ and R₁₄ are independently (C₁-C₂₄) alkyl and optionally have up to 6 double bonds, provided that at least one of R₈ and R₈ₐ is not hydrogen and that both R₈ and R₈ₐ are not R₁₁CO₂— or R₁₄COS— or combinations thereof, R₁₀ and R₁₀ₐ are independently, hydrogen, R₁₅CO₂—, R₁₆O—, R₁₇S—, R₁₈COS—, R₁₉CON—, R₂ONH—, R₂₁NR₂₂—, oxo, halogen, NH₂, —OH, or —SH and R₁₅, R₁₆, R₁₇, R₁₈, R₁₉, R₂₀, R₂₁ and R₂₂ are independently (C₁-C₇) alkyl provided that both R₁₀ and R₁₀ₐ are not halogen, NH₂, —OH, R₁₅CO₂—, R₁₈COS—, R₁₉CON—, R₂ONH—, R₂₁NR₂₂—, —SH or combinations thereof and that when either R₁₀ and R₁₀ₐ are oxo the other of R₁₀ and R₁₀ₐ is not defined,

### R₉ is

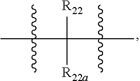

R₂₂ and R₂₂ₐ are independently, hydrogen, R₂₃CO₂—, R₂₄O—, R₂₅S—, R₂₆COS, R₂₇CON—, R₂₈NH—,R₂₈NR₂₉—, oxo, halogen, NH₂, —OH, or —SH and R₂₃, R₂₄, R₂₅, R₂₆, R₂₇, R₂₈, R₂₉ and R₃₀ are independently (C₁-C₇) alkyl provided that both R₂₂ and R₂₂ₐ are not halogen, R₂₃CO₂—, R₂₆COS, R₂₇CON—, R₂₈NH—,R₂₈NR₂₉—,NH₂, —OH, —SH or combinations thereof and that when either R₁₀ and R₁₀ₐ are oxo the other of R₁₀ and R_{10b} is not defined and m is an integer from 0 to 6. In other embodiments, m is 0, 1 or 2. In still other embodiments, m is 0 or 1. In still other embodiments, m is 0. In still other embodiments, R₁₀ and R₁₀ₐ are hydrogen. In still other embodiments, R₂₂ and R₂₂ₐ are hydrogen. In still other embodiments, R₁₀, R₁₀ₐ, R₂₂ and R₂₂ₐ are hydrogen. In still other embodiments, R₃ is

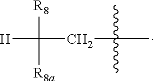

In still other embodiments, R₃ is

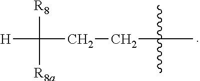

In still other embodiments, R₃ is acetyl, valyl, dipivoxil, bis(pivaloyloxymethyl) or disoproxil. In still other embodiments, R₃ is hexadecyloxypropyl, octadecyloxyethyl, or oleyloxyethyl. In still other embodiments, R₈ is R_{12O}, R₁₂ is (CH₂)ₜ—CH₃ and t is 0 to 24, In still other embodiments, t is 8, 10, 12, 13, 14, 15, 16, 17, 18, 19 or 20. In still other embodiments, t is 13, 14, 15, 16, 17, 18, 19 or 20. In still other embodiments, t is 15, 16, 17, 18, 19 or 20. In still other embodiments, t is 17, 18, 19 or 20. In still other embodiments, t is 15 or 17, 18.

In some embodiments, R₃ is (C₈-C₂₄) alkyl or substituted (C₈-C₂₄) alkyl. In other embodiments, R₃ has up to 6 double bonds.

In some embodiments, R₃ is C₈ alkyl, C₁₀ alkyl or (C₁₂-C₂₄) alkyl. In other embodiments, R₃ is (C₁₆-C₂₄) alkyl. In still other embodiments, R₃ is (C₁₇-C₂₂) alkyl. In still other embodiments, R₃ is (C₁₇-C₁₉) alkyl. In still other embodiments, R₃ is (C₁₇-C₂₂) alkanyl. In still other embodiments, R₃ is (C₁₇-C₁₉) alkanyl. In still other embodiments, R₃ is 7-methyl-octyl, 8-methyl-nonyl, 9-methyl-decyl, 10-methyl-undecyl, 11-methyl-dodecyl, 12-methyl-tridecyl, 13-methyl-tetradecyl, 14-methyl-pentadecyl, 15-methyl-hexadecyl, 16-methyl-heptadecyl, 17-methyl-octadecyl, 18-methyl-nonadecyl, 19-methyl-eicosyl, 20-methyl-heneicosyl, 21-methyl-docosyl, 22-methyl-tricosyl, 7-fluoro-octyl, 8-fluoro-nonyl, 9-fluoro-decyl, 10-fluoro-undecyl, 11-fluoro-dodecyl, 12-fluoro-tridecyl, 13-fluoro-tetradecyl, 14-fluoro-pentadecyl, 15-fluoro-hexadecyl, 16-fluoro-heptadecyl, 17-fluoro-octadecyl, 18-fluoro-nonadecyl, 19-fluoro-eicosyl, 20-fluoro-heneicosyl, 21-fluoro-docosyl or 22-fluoro-tricosyl.

In some embodiments, R₃ is n-C₁₄H₂₉O(CH₂)₃—, n-C₁₅H₃₁O(CH₂)₃—, n-C₁₆H₃₃O(CH₂)₃—, n-C₁₇H₃₅O(CH₂)₃— or n-C₁₈H₃₇O(CH₂)₃—.

In some embodiments, B is

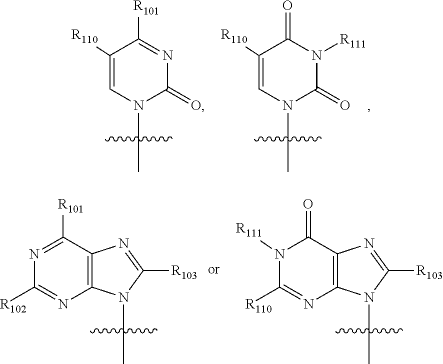

wherein R₁₁₀ is hydrogen, alkyl, substituted alkyl, —OH, halo, aryl or heteroaryl, R₁₁₁ is hydrogen or alkyl, R₁₀₁ is hydrogen, hydroxyl, halo, alkyl, —OR₁₀₄, —SR₁₀₅, —NR₁₀₆NH₂, —NR₁₀₇NHSO₂Me or —NR₁₀₈R₁₀₉, R₁₀₂ is hydrogen, alkyl, halo or —NR₁₀₈R₁₀₉, and R₁₀₃, R₁₀₄, R₁₀₅, R₁₀₆, R₁₀₇, R₁₀₈ and R₁₀₉ are independently hydrogen or alkyl and R₁₀₃ is hydrogen or alkyl. In some embodiments, R₁₁₀ is hydrogen or alkanyl. In still other embodiments, R₁₁₀ is hydrogen or methyl. In still other embodiments, R₁₀₈ and R₁₀₉ are independently hydrogen, alkanyl or cycloalkanyl. In still other embodiments, R₁₀₈ and R₁₀₉ are independently hydrogen, alkanyl or cycloalkanyl. In still other embodiments, R₁₀₈ is hydrogen, methyl or cyclopropyl. In still other embodiments, R₁₁₀ is hydrogen or alkanyl. In still other embodiments, R₁₁₀ is hydrogen or methyl. In still other embodiments, R₁₀₁ is hydrogen, hydroxyl, halo, alkyl, or —NR₁₀₈R₁₀₉, In still other embodiments, R₁₀₁ is hydrogen, alkyl, or —NR₁₀₈R₁₀₉. In still other embodiments, R₁₀₁ is methyl, NH₂ or —NR₁₀₈R₁₀₉. In still other embodiments, R₁₀₁ is —OR₁₀₄, —SR₁₀₅, —NR₁₀₆NH₂ or —NR₁₀₇NHSO₂Me. In still other embodiments, R₁₀₄, R₁₀₅, R₁₀₆ or R₁₀₇ are methyl. In still other embodiments, R₁₀₂ is hydrogen. In still other embodiments, R₁₀₃ is hydrogen.

In some embodiments, B is a pyrimidin-1-yl, pyrimidin-3-yl, purin-3-yl, purin-7-yl, or purin-9-yl. In other embodiments, B is thymin-1-yl, cytosin-1-yl, adenine-9-yl or guanine-9-yl. In still other embodiments, B is

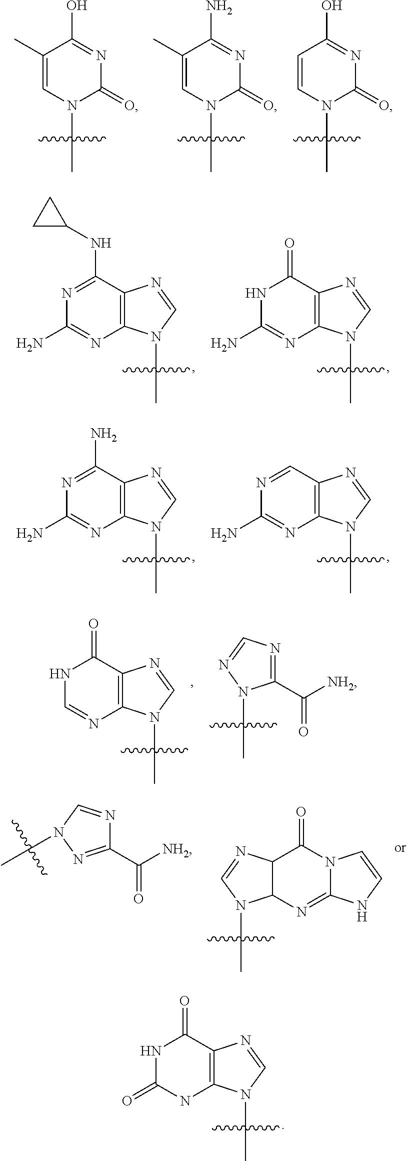

In some embodiments, B is

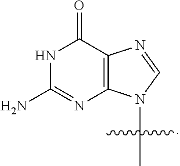 [SMILES: *n1cnc2c(=O)[nH]c(N)nc21]

In some embodiments, R₂ is alkyl or halo. In other embodiments, R₂ is methyl or fluoro.

In some embodiments, R₁ and R₂ together with the atoms to which they are bonded form a cycloalkyl or substituted cycloalkyl ring. In other embodiments, R₁ and R₂ together with the atoms to which they are bonded form a cyclopropyl or substituted cyclopropyl ring. In still other embodiments, R₁ and R₂ together with the atoms to which they are bonded form

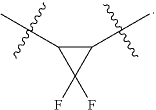 [SMILES: *C1C(*)C1(F)F]

In some embodiments, R₃ is n-C₁₆H₃₃O(CH₂)₃— and B is

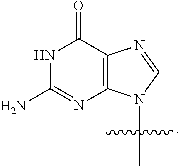 [SMILES: *n1cnc2c(=O)[nH]c(N)nc21]

In some embodiments, R₃ is n-C₁₆H₃₃O(CH₂)₃— and R₂ is alkyl or halo. In other embodiments, R₃ is n-C₁₆H₃₃O(CH₂)₃— and R₂ is methyl or fluoro. In still other embodiments, R₃ is n-C₁₆H₃₃O(CH₂)₃— and R₁ and R₂ together with the atoms to which they are bonded form a cycloalkyl or substituted cycloalkyl ring. In still other embodiments, R₃ is n-C₁₆H₃₃O(CH₂)₃— and R₁ and R₂ together with the atoms to which they are bonded form

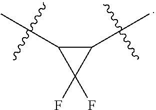 [SMILES: *C1C(*)C1(F)F]

In some embodiments, B is

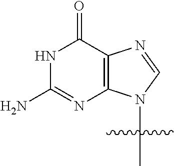 [SMILES: *n1cnc2c(=O)[nH]c(N)nc21]

and R₂ is alkyl or halo. In other embodiments, B is

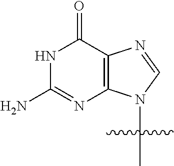 [SMILES: *n1cnc2c(=O)[nH]c(N)nc21]

and R₂ is methyl or fluoro. In still other embodiments, B is

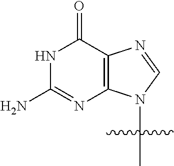 [SMILES: *n1cnc2c(=O)[nH]c(N)nc21]

and R₁ and R₂ together with the atoms to which they are bonded form a cycloalkyl or substituted cycloalkyl ring. In still other embodiments, B is

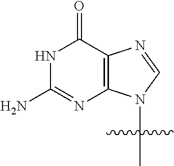 [SMILES: *n1cnc2c(=O)[nH]c(N)nc21]

and R₁ and R₂ together with the atoms to which they are bonded form

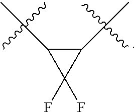 [SMILES: *C1C(*)C1(F)F]

In some embodiments, R₃ is n-C₁₆H₃₃O(CH₂)₃—, B is

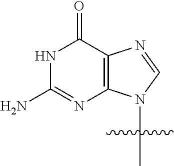 [SMILES: *n1cnc2c(=O)[nH]c(N)nc21]

and R₂ is alkyl or halo. In still other embodiments, R₃ is n-C₁₆H₃₃O(CH₂)₃—, B is

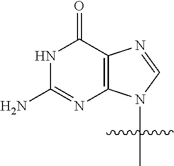 [SMILES: *n1cnc2c(=O)[nH]c(N)nc21]

and R₂ is methyl or fluoro. In still other embodiments, R₃ is n-C₁₆H₃₃O(CH₂)₃—, B is

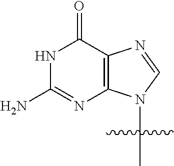 [SMILES: *n1cnc2c(=O)[nH]c(N)nc21]

and R₁ and R₂ together with the atoms to which they are bonded form a cycloalkyl or substituted cycloalkyl ring. In still other embodiments, R₃ is n-C₁₆H₃₃O(CH₂)₃—, B is

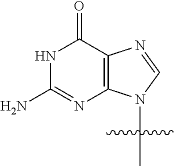 [SMILES: *n1cnc2c(=O)[nH]c(N)nc21]

and R₁ and R₂ together with the atoms to which they are bonded form

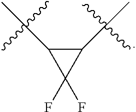 [SMILES: *C1C(*)C1(F)F]

In some embodiments, R₃ is n-C₁₆H₃₃O(CH₂)₃— and B is

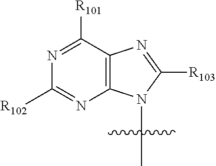

wherein R₁₀₁ is —OR₁₀₄, —SR₁₀₅, —NR₁₀₆NH₂ or —NR₁₀₇NHSO₂Me, R₁₀₂ is hydrogen, alkyl, halo or —NR₁₀₈R₁₀₉, R₁₀₃ is hydrogen or alkyl and R₁₀₄, R₁₀₅, R₁₀₆, R₁₀₇, R₁₀₈ and R₁₀₉ are independently hydrogen or alkyl and R₁ and R₂ are hydrogen. In other embodiments, R₁₀₄, R₁₀₅, R₁₀₆, R₁₀₇ are alkyl and R₁₀₂ is —NR₁₀₈R₁₀₉. In still other embodiments, R₁₀₄, R₁₀₅, R₁₀₆, R₁₀₇ are alkyl and R₁₀₂ is —NR₁₀₈R₁₀₉ and R₁₀₃ is hydrogen. In still other embodiments, R₁₀₄, R₁₀₅, R₁₀₆, R₁₀₇ are methyl or ethyl and R₁₀₂ is —NH₂ and R₁₀₃ is hydrogen. In still other embodiments, R₁₀₄ is methyl or ethyl, R₁₀₅, R₁₀₆, R₁₀₇ are methyl, R₁₀₂ is —NH₂ and R₁₀₃ is hydrogen. In some of the above embodiments, R₁ and R₂ are hydrogen. In some of the above embodiments, R₁ and R₂ are hydrogen in the compounds of Formula (I). In some of the above embodiments, R₂ is methyl or fluoro. In some of the above embodiments, R₁ and R₂ together with the atoms to which they are bonded form a cycloalkyl or substituted cycloalkyl ring. In some of the above embodiments, R₁ and R₂ together with the atoms to which they are bonded form a cyclopropyl or substituted cyclopropyl ring. In some of the above embodiments. R₁ and R₂ together with the atoms to which they are bonded form

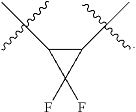 [SMILES: *C1C(*)C1(F)F]

In some of the above embodiments, R₁ and R₂ together with the atoms to which they are bonded form a cycloheteroalkyl or substituted cycloheteroalkyl ring. In some of the above embodiments, R₁ and R₂ together with the atoms to which they are bonded form

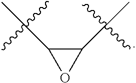 [SMILES: *C1OC1*]

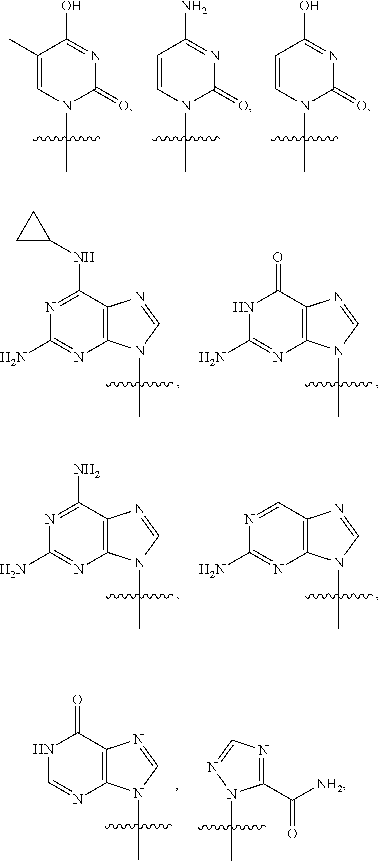

In some embodiments, B is

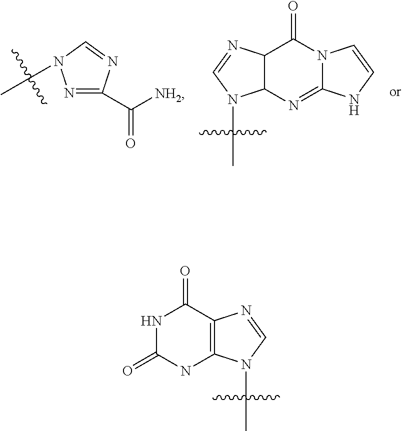 [SMILES: *n1cnc(C(N)=O)n1 ; *N1C=NC2C(=O)N3C=CNC3=NC21 ; "or" ; *n1cnc2c(=O)[nH]c(=O)[nH]c21]

and R₂ is alkyl or halo. In other embodiments, B is

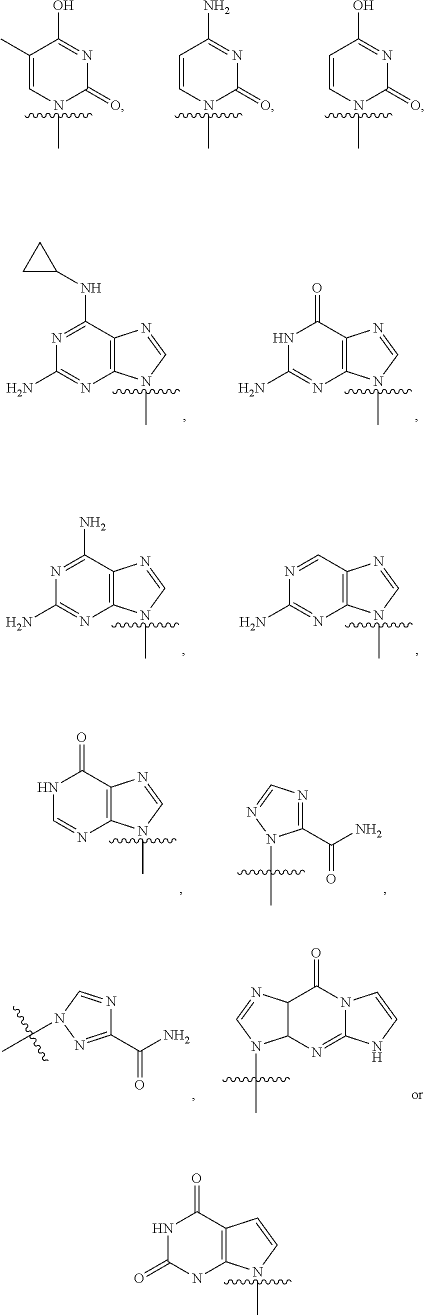

and R₂ is methyl or fluoro. In still other

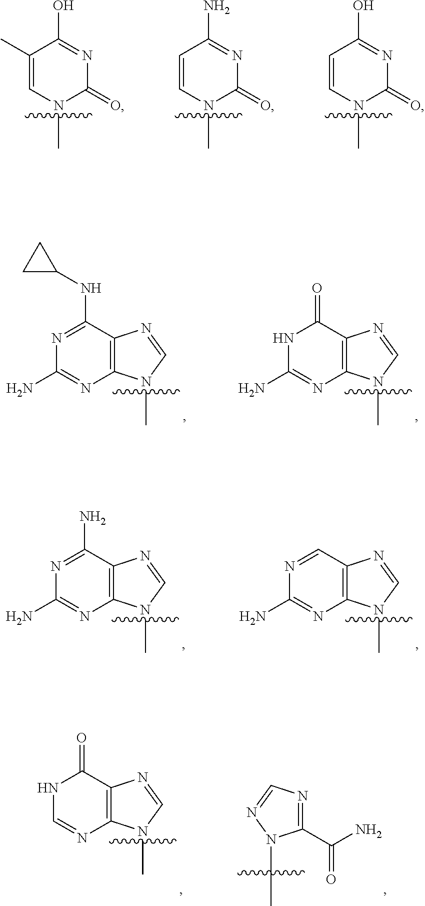

embodiments, B is

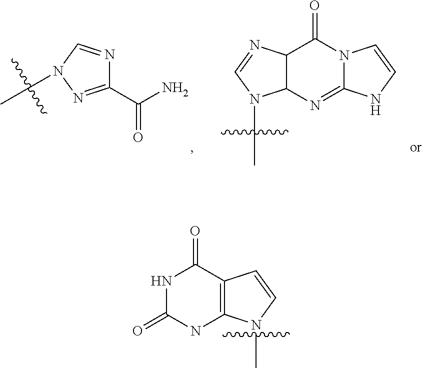

and R₁ and R₂ together with the atoms to which they are bonded form a cycloalkyl or substituted cycloalkyl ring. In still

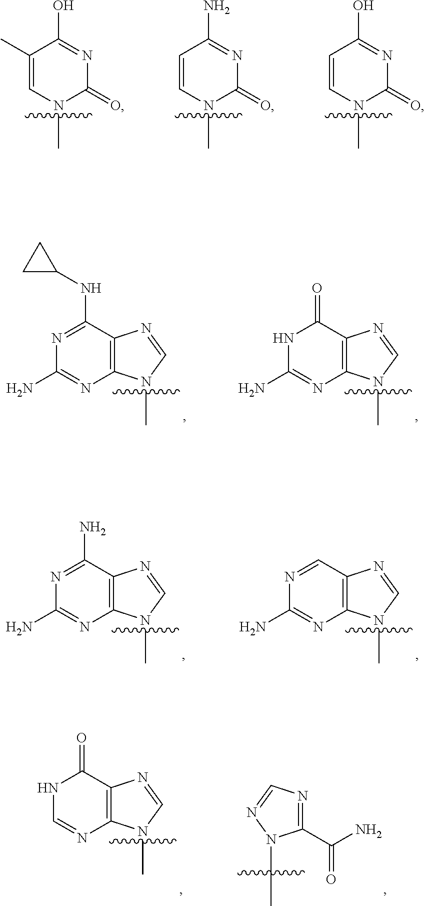

other embodiments, B is

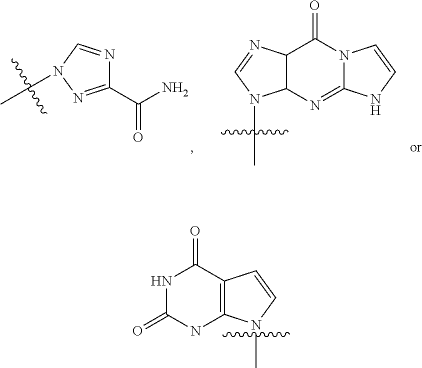

and R₁ and R₂ together with the atoms to which they are bonded form

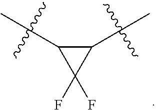 [SMILES: *C1C(*)C1(F)F]

In some embodiments, R₃ is n-C₁₆H₃₃O(CH₂)₃— and B

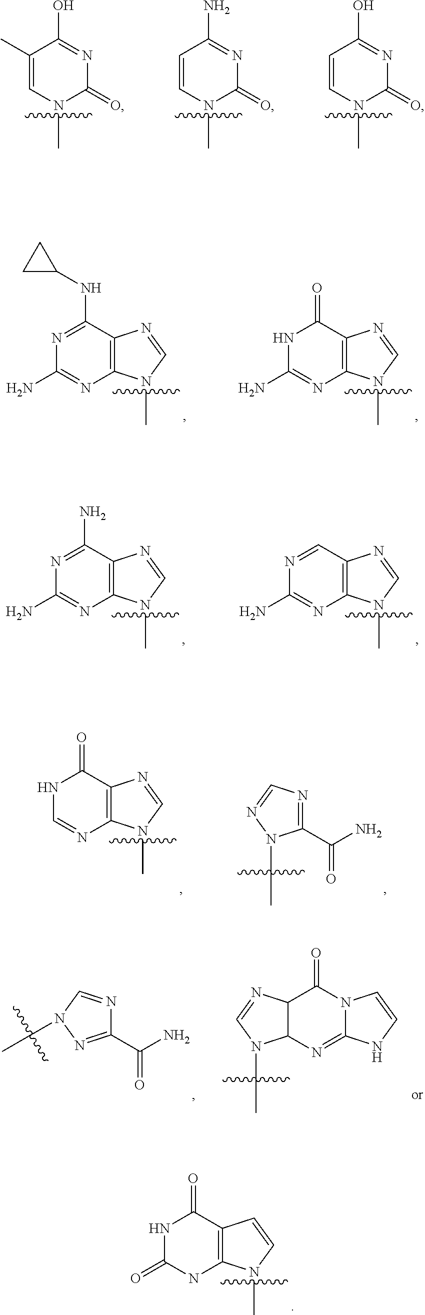

In other embodiments, R₂ is methyl or fluoro. In still other embodiments, R₁ and R₂ together with the atoms to which they are bonded form a cycloalkyl or substituted cycloalkyl ring. In still other embodiments, R₁ and R₂ together with the atoms to which they are bonded form a cyclopropyl or substituted cyclopropyl ring. In still other embodiments, R₁ and R₂ together with the atoms to which they are bonded form

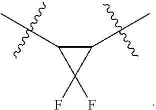 [SMILES: *C1C(*)C1(F)F]

In some embodiments, R₁ and R₂ together with the atoms to which they are bonded form a cycloheteroalkyl or substituted cycloheteroalkyl ring. In other embodiments, R₁ and R₂ together with the atoms to which they are bonded form

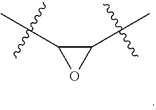 [SMILES: *C1OC1*]

In some of all of the above embodiments, n is 0. In other of embodiments, n is 0 when B is

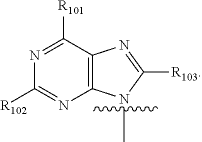

In other embodiments, n is 0 when R₃ is n-C₁₆H₃₃O(CH₂)₃— and B is

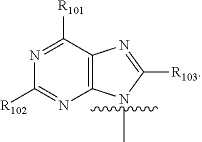

In still other embodiments, n is 0 when B is

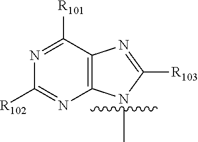

and R₁ and R₂ are hydrogen. In still other embodiments, n is 0 when B is

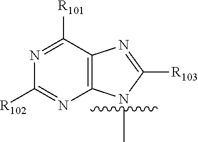

R₁₀₂ is NH₂, R₁₀₃ is hydrogen and R₁ and R₂ are hydrogen. In still other embodiments, n is 0 when R₃ is n-C₁₆H₃₃O(CH₂)₃—, B is

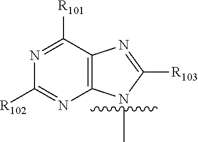

and R₁ and R₂ are hydrogen. In still other embodiments, n is 0 when R₃ is n-C₁₆H₃₃O(CH₂)₃—, B is

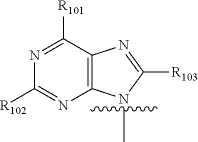

R₁₀₂ is NH₂, R₁₀₃ is hydrogen and R₁ and R₂ are hydrogen.

In a second aspect, compounds of Formula (III):

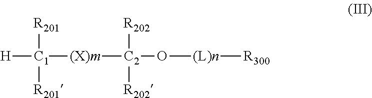

or salts, solvates or hydrates thereof are provided wherein: R₂₀₁ and R_{201′}, are independently hydrogen, —OR₂₀₃, —OC(O)R₂₀₄, —SR₂₀₅ or —SC(O)R₂₀₆, R₂₀₃, R₂₀₄, R₂₀₅ or R₂₀₆ are independently alkyl, provided that both R₂₀₁ and R_{201′}, are not OC(O)R₂₀₄, —SC(O)R₂₀₆, or combinations thereof, each R₂₀₂ and R_{202′}, are independently —OR₂₀₇, —OC(O)R₂₀₈, —SR₂₀₉, —SC(O)_{R210}, —NR₂₁₁R₂₁₂, or —NC(O)R₂₁₃, R₂₀₇, R₂₀₉, R₂₁₁ or R₂₁₂ are independently hydrogen or alkyl and R₂₀₈, R₂₁₀ and R₂₁₃ are independently alkyl, provided that both R₂₀₁ and R_{201′}, attached to the same carbon atom are not both —OC(O)R₂₀₈, —SC(O)R₂₁₀, —NR₂₁₁R₂₁₂, or —NC(O)R₂₁₃ and R₃₀₀ is a phosphonate containing a nucleotide phosphonate or an antiviral phosphonate which substitutes the pyrimidine base R₃₀₁ for the purine or pyrimidine base of the nucleotide or nucleoside or the antiviral phosphonate

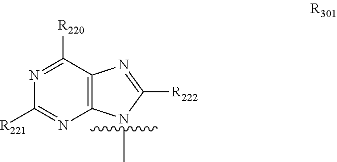

wherein R₂₂₀ is hydrogen, hydroxyl, halo, alkyl, —OR₂₂₄, —SR₂₂₅, —NR₂₂₆NH₂ or —NR₂₂₇NHSO₂Me or —NR₂₂₈R₂₂₉, R₂₂₁ is hydrogen, alkyl, halo or —NR₂₂₈R₂₂₉, R₂₂₂ is hydrogen or alkyl and R₂₂₄, R₂₂₅, R₂₂₆NH₂, R₂₂₇, R₂₂₈ and R₂₂₉ are hydrogen or alkyl, X is —(R₂₀₂)C(R_{202′})—, L is a valence bond or a bifunctional linking molecule of the formula -J-(CR₂₃₀)ₜ-G- wherein t is an integer from 1 to 24, J and G are independently —O—, —S—, —C(O)O— or —NH— and R₂₃₀ is hydrogen, alkyl or substituted alkyl, m is an integer from 0 to 6 and n is 0 or 1. In some embodiments, R₂₀₁ and R_{201′}, are not both hydrogen.

In some embodiments, R₃₀₀ is derived from the compounds depicted

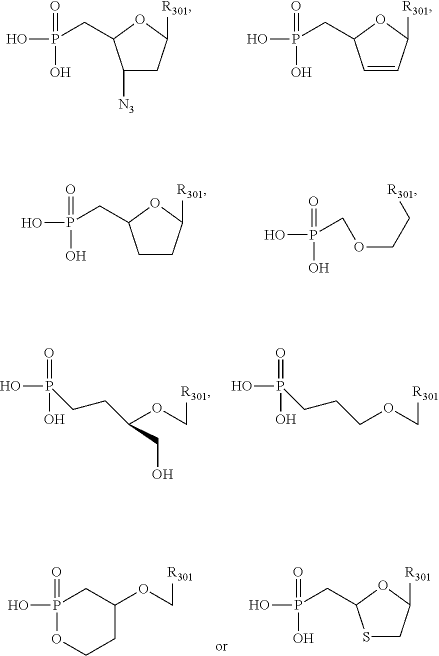

above by replacement of one of the hydrogen atoms on the hydroxyl attached to the phosphorus atom with a bond.

In some embodiments, the nucleotide phosphonate is similarly derived from ddA, ddI, ddG, L-FMAU, DXG, DAPD, L-dA, L-dl, L-(d)T, L-dC, L-dG, FTC, peniciclor, 2-chloro-deoxyadenosine, cytarabine, fluorouiridine, fluordeoxyuridine, gemcitabine, cladribine, fludaribine, pentostatin, ara-G, ara-A and ara-U.

In some embodiments, the antiviral phosphonate is similarly derived from cidofovir, cyclic cidofovir, adefovir, tenofovir and valamociclovir.

### Preparation of the Compounds

Exemplary methods for the preparation of nucleoside phosphonates and esters thereof for use in the compositions and methods provided herein are described below and in the Examples but other methods known in the art can be used to prepare the nucleoside phosphonates and esters thereof provided herein.

Scheme I below describes a general scheme for the synthesis of trisubstituted olefinic compounds of Formula (I) or Formula (II) where X, n R₁, R₂ and R₃ are as previously defined. Those of skill in the art will appreciate that many different reagents may be employed to effect the transformations below and that many different protecting groups can be used in compounds A-J below.

Referring now to Scheme I below, Compound A, which is either commercially available or can be readily prepared from known compounds using conventional methods is selectively monoprotected to provide compound B. The free alcohol group in Compound B is oxidized to yield the carbonyl compound C. Horner Emmons reaction or other method known in the art can be used to yield compounds D and E either as a mixture which is then resolved or stereoselectively. For ease of illustration, only compound E will be shown in subsequent steps of Scheme 1, but it should be understood that compound D can be similarly processed. Reduction of the ester functionality which can be accomplished by a number of methods provides the alcohol F which is then differentially protected to yield compound G. Selective removal of the protecting group P′ yields primary alcohol H which is then converted to a leaving group to provide compound I. Phosphite displacement then yields the phosphonate J (R′ and R″ are alkyl or aryl) which is then deprotected to provide alcohol K. The alcohol K is displaced with, for example, a purine base derivative, (those of skill in the art will be aware that a pyrimidine derivative can also be attached at this point using similar methods) to provide the protected vinyl phosphonate L, hydrolysis of which provides the phosphoric acid M. Selective attachment of a lipophilic sidechain provides the phosphonate N which is hydrolyzed to yield the vinyl purine phosphonate O.

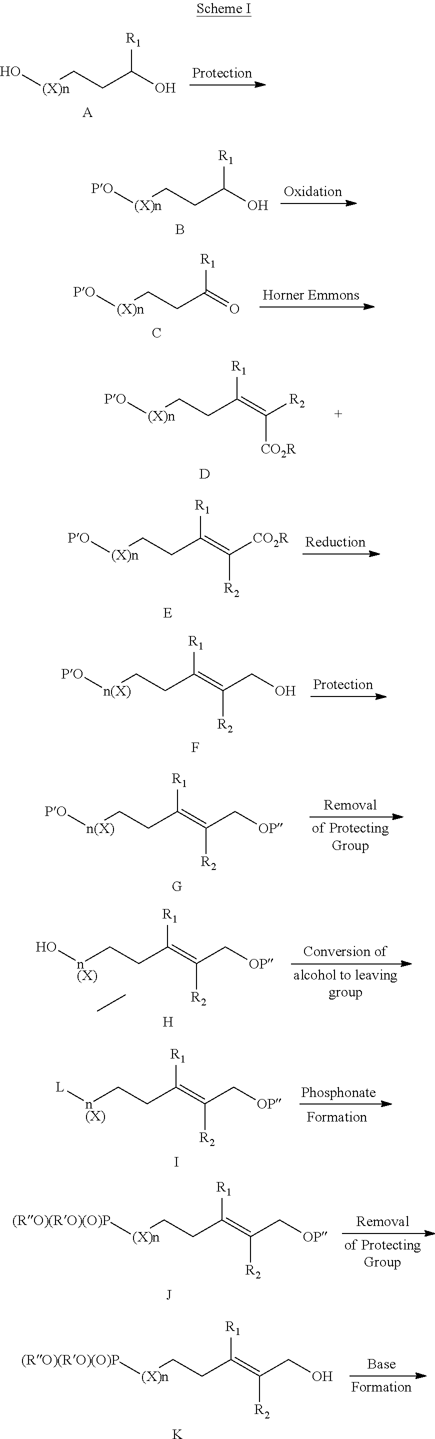

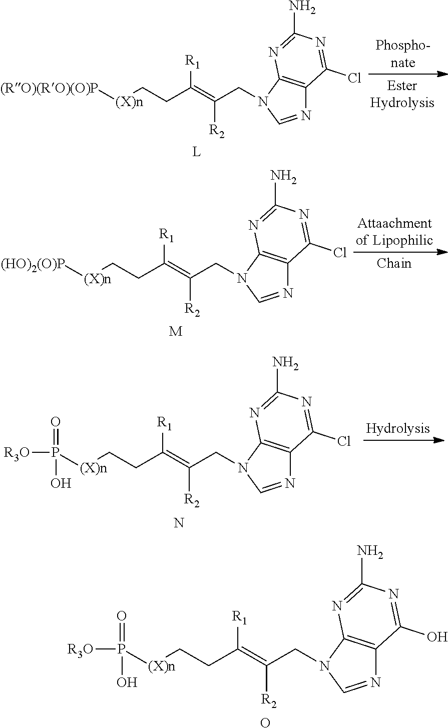

Scheme II below describes a general scheme for the synthesis of cyclic compounds of Formula (I) or Formula (II) where X, n R₁, R₂ and R₃ are as previously defined. Those of skill in the art will appreciate that many different reagents may be employed to effect the transformations below and that many different protecting groups can be used in compounds A′-H′ below.

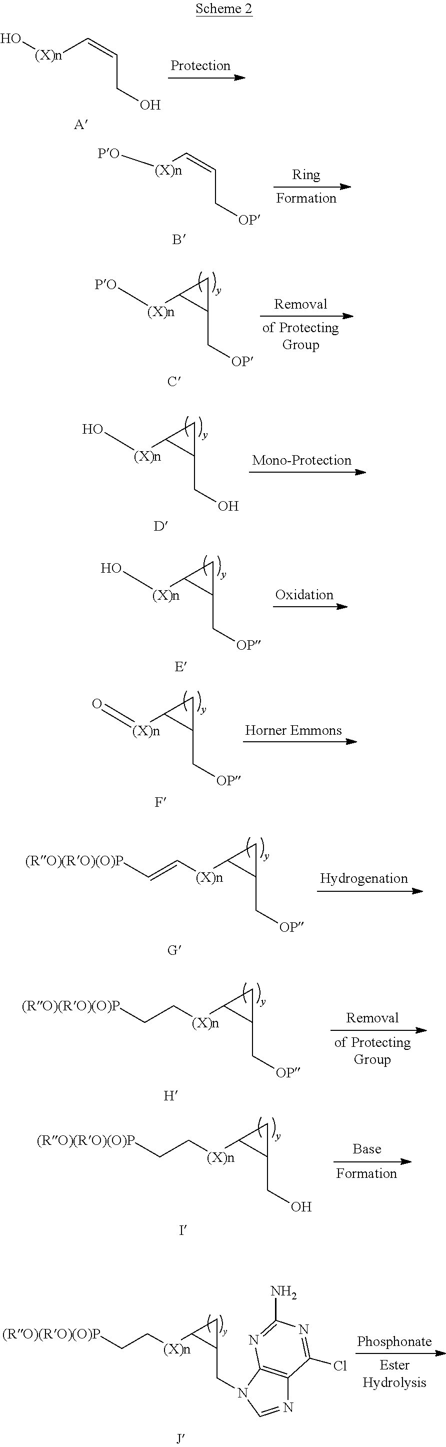

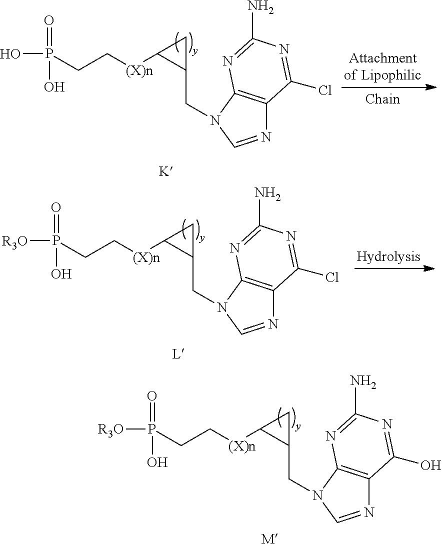

Referring now to Scheme II above, Compound A′, which is either commercially available or can be readily prepared from known compounds using conventional methods, is protected to provide compound B′. The olefin can be converted in one step by conventional methods to a cyclic compound when y is 1 or 2 to provide compound C′. Those of skill in the art will appreciate that compounds of Formula C′ can be prepared via conventional methods from pre-existing ring compounds where y is 3-8. Removal of the protecting group P′ yields the diol D′ which is monoprotected to yield alcohol E′ and oxidized to give the carbonyl compound F′. Horner Emmons reaction, for example, yields the vinyl phosphonate G′ (R′ and R″ are alkyl or aryl) which is then hydrogenated to provide the saturated phosphonate H′. The protecting P″ is removed to provide the alcohol I′ which is then is then displaced with, for example, a purine base derivative, (those of skill in the art will be aware that a pyrimidine derivative can also be attached at this point using similar methods) to provide the phosphonate J′, hydrolysis of which provide the phosphoric acid K′. Selective attachment of a lipophilic sidechain provides the phosphonate L′ which is hydrolyzed to yield the purine phosphonate M′.

Scheme III below, describes a general scheme for the synthesis of disubstituted olefinic compounds of Formula (I) or Formula (II) where X, n R₁, R₂, R₃ and R₁₀₁ are as previously defined. Those of skill in the art will appreciate that many different reagents may be employed to effect the transformations below.

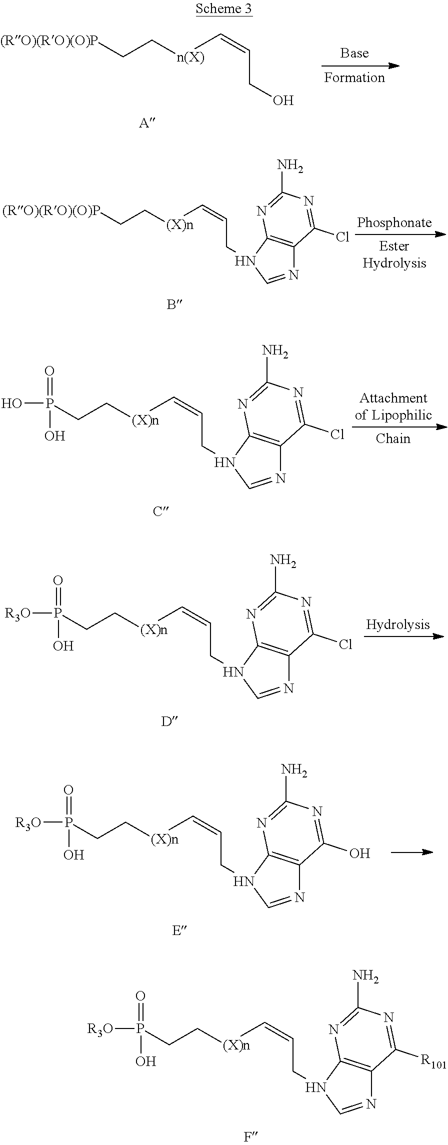

Referring now to Scheme III above, Compound A″, (R′ and R″ are alkyl or aryl) which is either commercially available or can be readily prepared from known compounds using conventional methods, is converted via alcohol displacement to the phosphonate B″, hydrolysis of which provide the phosphoric acid C″. Selective attachment of a lipophilic sidechain provides the phosphonate D″ which is hydrolyzed to yield the phosphonate E′. Phosphonate E′ can then be converted by known methods to various purine derivatives F″ where R₁₀₁ is as previously defined.

The compounds of Formula (III) may be made by the methods described in Hostetler et al., U.S. Pat. No. 7,098,197. Other methods of making the compounds of Formula (III) will also be apparent to those of skill in the art.

### Pharmaceutical Compositions and Methods of Administration

The pharmaceutical compositions provided herein contain therapeutically effective amounts of one or more of the compounds provided herein that are useful in the prevention, treatment, or amelioration of one or more of the symptoms of diseases or disorders associated with viral infections and inappropriate cell proliferation and a pharmaceutically acceptable vehicle. Pharmaceutical vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

The compositions contain one or more compounds provided herein. The compounds are, in some embodiments, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In some embodiments, the compounds described above are formulated into pharmaceutical compositions using techniques and procedures well known in the art (see, e.g., Ansel Introduction to Pharmaceutical Dosage Forms, Seventh Edition (1999).

In the compositions, effective concentrations of one or more compounds or pharmaceutically acceptable derivatives thereof is (are) mixed with a suitable pharmaceutical vehicle. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats, prevents, or ameliorates one or more of the symptoms of diseases or disorders associated with associated with viral infections or inappropriate cell proliferation. In some embodiments, the compositions are formulated for single dosage administration. To formulate a composition, the weight fraction of a compound is dissolved, suspended, dispersed or otherwise mixed in a selected vehicle at an effective concentration such that the treated condition is relieved, prevented, or one or more symptoms are ameliorated.

The active compound is included in the pharmaceutically acceptable vehicle in an amount sufficient to exert a therapeutically useful effect in the absence of undesirable side effects on the patient treated. The therapeutically effective concentration may be determined empirically by testing the compounds in in vitro and in vivo systems well known to those of skill in the art and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with viral infections or inappropriate cell proliferation, as described herein.

In some embodiments, a therapeutically effective dosage should produce a serum concentration of active ingredient of from about 0.1 ng/ml to about 50-100 μg/ml. The pharmaceutical compositions, in other embodiments, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in some embodiments from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using co-solvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in some embodiments, formulated and administered in unit-dosage forms or multiple-dosage forms. Unit-dose forms as used herein refer to physically discrete units suitable for human and animal subjects and packaged individually as is known in the art. Each unit-dose contains a predetermined quantity of the therapeutically active compound sufficient to produce the desired therapeutic effect, in association with the required pharmaceutical vehicle. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a vehicle, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975 or later editions thereof.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

In certain embodiments, the compositions are lactose-free compositions containing excipients that are well known in the art and are listed, for example, in the U.S. Pharmacopeia (USP) 25-NF20 (2002). In general, lactose-free compositions contains active ingredients, a binder/filler, and a lubricant in pharmaceutically compatible and pharmaceutically acceptable amounts. Particular lactose-free dosage forms contain active ingredients, microcrystalline cellulose, pre-gelatinized starch, and magnesium stearate.

Further provided are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are generally packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms such as for example, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable vehicles used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example, propylene carbonate, vegetable oils or triglycerides, is in some embodiments encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid vehicle, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

Parenteral administration, in some embodiments characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable vehicles used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; 6,699,500 and 6,740,634. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In some embodiments, a pump may be used (see, Sefton, CRC Crit. Ref Biomed. Eng. 14:201 (1987); Buchwald et al., Surgery 88:507 (1980); Saudek et al., N. Engl. J. Med. 321:574 (1989). In other embodiments, polymeric materials can be used. In other embodiments, a controlled release system can be placed in proximity of the therapeutic target, i.e., thus requiring only a fraction of the systemic dose (see, e.g., Goodson, Medical Applications of Controlled Release, vol. 2, pp. 115-138 (1984). In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, an antioxidant, a buffer and a bulking agent. In some embodiments, the excipient is selected from dextrose, sorbital, fructose, corn syrup, xylitol, glycerin, glucose, sucrose and other suitable agent. The solvent may contain a buffer, such as citrate, sodium or potassium phosphate or other such buffer known to those of skill in the art at, at about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in some embodiments, have diameters of less than 50 microns, in other embodiments less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

For nasal administration, the preparation may contain an esterified phosphonate compound dissolved or suspended in a liquid carrier, in particular, an aqueous carrier, for aerosol application. The carrier may contain solubilizing agents such as propylene glycol, surfactants, absorption enhancers such as lecithin or cyclodextrin, or preservatives.

These solutions, particularly those intended for ophthalmic use, may be formulated as 0.01%-10% isotonic solutions, pH about 5-7, with appropriate salts.

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm. Tablets and capsules for rectal administration are manufactured using the same pharmaceutically acceptable substance and by the same methods as for formulations for oral administration.

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In some embodiments, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The compounds or pharmaceutically acceptable derivatives may be packaged as articles of manufacture containing packaging material, a compound or pharmaceutically acceptable derivative thereof provided herein, which is effective for treatment, prevention or amelioration of one or more symptoms of diseases or disorders associated with viral infections or inappropriate cell proliferation, within the packaging material, and a label that indicates that the compound or composition, or pharmaceutically acceptable derivative thereof, is used for the treatment, prevention or amelioration of one or more symptoms of diseases or disorders associated with viral infections or inappropriate cell proliferation.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder associated with viral infections or inappropriate cell proliferation.

### Dosages

In human therapeutics, the physician will determine the dosage regimen that is most appropriate according to a preventive or curative treatment and according to the age, weight, stage of the disease and other factors specific to the subject to be treated. The pharmaceutical compositions, in another embodiment, should provide a dosage of from about 0.001 mg to about 2000 mg of compound per kilogram of body weight per day. Pharmaceutical dosage unit forms are prepared, e.g., to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The amount of active ingredient in the formulations provided herein, which will be effective in the prevention or treatment of a disorder or one or more symptoms thereof, will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject.

Exemplary doses of a formulation include milligram or microgram amounts of the active compound per kilogram of subject or sample weight (e.g., from about 1 micrograms per kilogram to about 50 milligrams per kilogram, from about 10 micrograms per kilogram to about 30 milligrams per kilogram, from about 100 micrograms per kilogram to about 10 milligrams per kilogram, or from about 100 microgram per kilogram to about 5 milligrams per kilogram).

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

### Evaluation of the Activity of the Compounds

The activity of the compounds as antivirals can be measured in standard assays known in the art. Exemplary assays include, but are not limited to, plaque reduction assay in HFF cells, DNA reduction assay in MRC-5 cells, p24 reduction assay in MT-2 cells, CPE assay in HFF cells and EBV Elisa assay in Daudi cells.

### Methods of Use of the Compounds and Compositions

Methods of treating, preventing, or ameliorating one or more symptoms of diseases associated with viral infections or inappropriate cell proliferation using the compounds and compositions are provided. In practicing the methods, effective amounts of the compounds or compositions containing therapeutically effective concentrations of the compounds are administered. In certain embodiments, the methods provided herein are for the preventing, or ameliorating one or more symptoms of diseases associated with viral infections, including, but not limited to influenza; hepatitis B and C virus; cytomegalovirus (CMV); herpes infections, such as those caused by Varicella zoster virus, Herpes simplex virus types 1 & 2, Epstein-Barr virus, Herpes type 6 (HHV-6) and type 8 (HHV-8); Varicella zoster virus infections such as shingles or chicken pox; Epstein Barr virus infections, including, but not limited to infectious mononucleosis/glandular fever; retroviral infections including, but not limited to SIV, HIV-1 and HIV-2; ebola virus; adenovirus and papilloma virus.

In further embodiments, the methods provided herein are for treating, preventing, treating, or ameliorating one or more symptoms of diseases associated with viral infections caused by orthopox viruses, such as variola major and minor, vaccinia, smallpox, cowpox, camelpox, and monkeypox. In certain embodiments, the disease is drug resistant hepatitis B.

In certain embodiments, the methods provided herein are for treating, preventing, or ameliorating one or more symptoms of diseases associated with cell proliferation, including, but not limited to cancers. Examples of cancers include, but are not limited to, lung cancer, head and neck squamous cancers, colorectal cancer, prostate cancer, breast cancer, acute lymphocytic leukemia, adult acute myeloid leukemia, adult non Hodgkin's lymphoma, brain tumors, cervical cancers, childhood cancers, childhood sarcoma, chronic lymphocytic leukemia, chronic myeloid leukemia, esophageal cancer, hairy cell leukemia, kidney cancer, liver cancer, multiple myeloma, neuroblastoma, oral cancer, pancreatic cancer, primary central nervous system lymphoma, and skin cancer.

### Combination Therapy

The compounds and compositions provided herein may also be used in combination with one or more other active ingredients. In certain embodiments, the compounds may be administered in combination, or sequentially, with another therapeutic agent. Such other therapeutic agents include those known for treatment, prevention, or amelioration of one or more symptoms associated with viral infections or inappropriate cell proliferation. Such therapeutic agents include, but are not limited to, antiviral agents and anti-neoplastic agents.

Recently, it has been demonstrated that the efficacy of a drug against HIV infection can be prolonged, augmented, or restored by administering the compound in combination or alternation with a second, and perhaps third, antiviral compound that induces a different mutation from that caused by the principle drug. Alternatively, the pharmacokinetics, biodistribution, or other parameter of the drug can be altered by such combination or alternation therapy.

In certain embodiments, provided herein are methods of treatment of prevention that encompass administration of a second agent effective for the treatment or prevention of viral infection, such as HIV and/or HCV infection. The second agent can be any agent known to those of skill in the art to be effective for the treatment, prevention or amelioration of viral infections, such as the HIV and/or HCV infection. The second agent can be a second agent presently known to those of skill in the art, or the second agent can be second agent later developed for the treatment, prevention or amelioration of viral infections. In certain embodiments, the second agent is presently approved for the treatment or prevention of HIV and/or HCV.

In certain embodiments, a compound provided herein is administered in combination with one second agent. In further embodiments, a second agent is administered in combination with two second agents. In still further embodiments, a second agent is administered in combination with two or more second agents.

The second antiviral agent for the treatment of HIV, in one embodiment, can be a reverse transcriptase inhibitor (a “RTI”), which can be either a synthetic nucleoside (a “NRTI”) or a non-nucleoside compound (a “NNRTI”). In an alternative embodiment, in the case of HIV, the second (or third) antiviral agent can be a protease inhibitor. In other embodiments, the second (or third) compound can be a pyrophosphate analog, or a fusion binding inhibitor.

In some embodiments, compounds for combination or alternation therapy for the treatment of HBV include, but are not limited to 3TC, FTC, L-FMAU, interferon, β-D-dioxolanyl-guanine (DXG), β-D-dioxolanyl-2,6-diaminopurine (DAPD), and β-D-dioxolanyl-6-chloropurine (ACP), famciclovir, penciclovir, BMS-200475, bis pom PMEA (adefovir, dipivoxil); lobucavir, ganciclovir, and ribavarin.

In other embodiments, examples of antiviral agents that can be used in combination or alternation with the compounds disclosed herein for HIV therapy include cis-2-hydroxymethyl-5-(5-fluorocytosin-1-yl)-1,3-oxathiolane (FTC); the (-;)-enantiomer of 2-hydroxymethyl-5-(cytosin-1-yl)-1,3-oxathiolane (3TC); carbovir, acyclovir, foscarnet, interferon, AZT, DDI, DDC, D4T, CS-87 (3′-azido-2′,3′-dideoxy-uridine), and β-D-dioxolane nucleosides such as β-D-dioxolanyl-guanine (DXG), β-D-dioxolanyl-2,6-diaminopurine (DAPD), and β-D-dioxolanyl-6-chloropurine (ACP), MKC442 (6-benzyl-1-(ethoxymethyl)-5-isopropyl uracil.

The protease inhibitors include crixivan (Merck), nelfinavir (Agouron), ritonavir (Abbott), saquinavir (Roche), DMP-266 (Sustiva) and DMP-450 (DuPont Merck).

Further compounds that can be administered in combination or alternation with any of the compounds provided herein include (1S,4R)-4-[2-amino-6-cyclopropyl-amino)-9H-purin-9-yl]-2-cyclopentene-1-methanol succinate (“1592”, a carbovir analog); 3TC; -β-L-2′,3′-dideoxy-3′-thiacytidine; a-APA R18893: a-nitro-anilino-phenylacetamide; A-77003; C2 symmetry-based protease inhibitor; A-75925: C2 symmetry-based protease inhibitor; AAP-BHAP: bisheteroarylpiperazine analog; ABT-538: C2 symmetry-based protease inhibitor; AzddU:3′-azido-2′,3′-dideoxyuridine; AZT: 3′-azido-3′-deoxythymidine; AZT-p-ddI:3′-azido-3′-deoxythymidilyl-(5′,5′)-2′,3′-dideoxyinosinic acid; BHAP: bisheteroarylpiperazine; BILA 1906: N-{1S-[[[3-[2S-{(1,1-dimethylethyl)amino]carbonyl}-4R-]3-pyridinylmethyl)thio]-1-piperidinyl]-2R-hydroxy-1S-(phenylmethyl)propyl]amino]carbonyl]-2-methylpropyl}-2-quinolinecarboxamide; BILA 2185: N-(1,1-dimethylethyl)-1-[2S-[[2-2,6-dimethyphenoxy)-1-oxoethyl]amino]-2R-hydroxy-4-phenylbutyl]4R-pyridinylthio)-2-piperidine-carboxamide; BM+51.0836: thiazolo-isoindolinone derivative; BMS 186,318: aminodiol derivative HIV-1 protease inhibitor; d4API: 9-[2,5-dihydro-5-(phosphonomethoxy)-2-furanyl]adenine; d4C: 2′,3′-didehydro-2′,3′-dideoxycytidine; d4T: 2′,3′-didehydro-3′-deoxythymidine; ddC; 2′,3′-dideoxycytidine; ddI: 2′,3′-dideoxyinosine; DMP-266: a 1,4-dihydro-2H-3,1-benzoxazin-2-one; DMP-450: {[4R-(4-a,5-a,6-b,7-b)]-hexahydro-5,6-bis(hydroxy)-1,3-bis(3-amino)phenyl]methyl)-4,7-bis(phenylmethyl)-2H-1,3-diazepin-2-one}-bismesylate; DXG: (-;)-β-D-dioxolane-guanosine; EBU-dM:5-ethyl-1-ethoxymethyl-6-(3,5-dimethylbenzyl)uracil; E-EBU: 5-ethyl-1-ethoxymethyl-6-benzyluracil; DS: dextran sulfate; E-EPSeU:1-(ethoxymethyl)-(6-phenylselenyl)-5-ethyluracil; E-EPU: 1-(ethoxymethyl)-(6-phenyl-thio)-5-ethyluracil; FTC:β-2′,3′-dideoxy-5-fluoro-3′-thiacytidine (Triangle); HBY097:S-4-isopropoxycarbonyl-6-methoxy-3-(methylthio-methyl)-3,4-dihydroquinoxalin-2(1H)-thione; HEPT:1-[(2-hydroxyethoxy)methyl]-6-(phenylthio)thymine; HIV-1:human immunodeficiency virus type 1; JM2763: 1,1′-(1,3-propanediyl)-bis-1,4,8,11-tetraazacyclotetradecane; JM3100:1,1′-[1,4-phenylenebis-(methylene)]-bis-1,4,8,11-tetraazacyclotetradecane; KNI-272: (2S,3S)-3-amino-2-hydroxy-4-phenylbutyric acid-containing tripeptide; L-697,593; 5-ethyl-6-methyl-3-(2-phthalimido-ethyl)pyridin-2(1H)-one; L-735,524:hydroxy-aminopentane amide HIV-1 protease inhibitor; L-697,661: 3-{[(-4,7-dichloro-1,3-benzoxazol-2-yl)methyl]amino}-5-ethyl-6-methylpyridin-2(1H)-one; L-FDDC: (-;)-β-L-5-fluoro-2′,3′-dideoxycytidine; L-FDOC:(-;)-β-L-5-fluoro-dioxolane cytosine; MKC442:6-benzyl-1-ethoxymethyl-5-isopropyluracil (1-EBU); Nevirapine: 1′-cyclopropyl-5,11-dihydro-4-methyl-6H-dipyridol[3,2-b:2′,3′-e]diazepin-6-one; NSC648400:1-benzyloxymethyl-5-ethyl-6-(alpha-pyridylthio)uracil (E-BPTU); P9941: [2-pyridylacetyl-IlePheAla-y(CHOH)]2; PFA: phosphonoformate; PMEA: 9-(2-phosphonylmethoxyethyl)adenine; PMPA: (R)-9-(2-phosphonyl-methoxypropyl)adenine; Ro 31-8959: hydroxyethylamine derivative HIV-1 protease inhibitor; RPI-312: peptidyl protease inhibitor, 1-[(3s)-3-(n-alpha-benzyloxycarbonyl)-1-asparginyl)-amino-2-hydroxy-4-phenylbutyryl]-n-tert-butyl-1-proline amide; 2720: 6-chloro-3,3-dimethyl-4-(isopropenyloxycarbonyl)-3,4-dihydro-quinoxalin-2(1H)thione; SC-52151: hydroxyethylurea isostere protease inhibitor; SC-55389A: hydroxyethyl-urea isostere protease inhibitor; TIBO R82150: (+)-(5S)-4,5,6,7-tetrahydro-5-methyl-6-(3-methyl-2-butenyl)imidazo[4,5,1-jk][1,4]-benzodiazepin-2(1H)-thione; TIBO 82913: (+)-(5S)-4,5,6,7,-tetrahydro-9-chloro-5-methyl-6-(3-methyl-2-butenyl)imidazo[4,5,1jk]-[1,4]benzo-diazepin-2(1H)-thione; TSAO-m3T:[2′,5′-bis-β-(tert-butyldimethylsilyl)-3′-spiro-5′-(4′-amino-1′,2′-oxathiole-2′,2′-dioxide)]-b-D-pentofiaranosyl-N-3-methylthymine; U90152:1-[3-[(1-methylethyl)-amino]-2-pyridinyl]-4-[[5-[(methylsulphonyl)-amino]-1H -indol-2-yl]carbonyl]-piperazine; UC: thiocarboxanilide derivatives (Uniroyal); UC-781=N-[4-chloro-3-(3-methyl-2-butenyloxy)phenyl]-2-methyl-3-furancarbothioamide; UC-82=N-[4-chloro-3-(3-methyl-2-butenyloxy)phenyl]-2-methyl-3-thiophenecarbothioamide; VB 11,328: hydroxyethyl-sulphonamide protease inhibitor; VX-478:hydroxyethylsulphonamide protease inhibitor; XM 323: cyclic urea protease inhibitor.

In certain embodiments, suitable second agents include small-molecule, orally bioavailable inhibitors of the HCV enzymes, nucleic-acid-based agents that attack viral RNA, agents that can modulate the host immune response. Exemplary second agents include: (i) current approved therapies (peg-interferon plus ribavirin), (ii) HCV-enzyme targeted compounds, (iii) viral-genome-targeted therapies (e.g., RNA interference or RNAi), and (iv) immunomodulatory agents such as ribavirin, interferon (INF) and Toll-receptor agonists.

In certain embodiments, the second agent is a modulator of the NS3-4A protease. The NS3-4A protease is a heterodimeric protease, comprising the amino-terminal domain of the NS3 protein and the small NS4A cofactor. Its activity is essential for the generation of components of the viral RNA replication complex.

One useful NS3-4A protease inhibitor is BILN 2061 (Ciluprevir; Boehringer Ingelheim), a macrocyclic mimic of peptide product inhibitors. Although clinical trials with BILN 2061 were halted (preclinical cardiotoxicity), it was the first NS3 inhibitor to be tested in humans. See Lamarre et al., 2003, Nature 426:186-189, the contents of which are hereby incorporated by reference in their entirety.

Another useful NS3-4A protease inhibitor is VX-950 (Vertex/Mitsubishi), a protease-cleavage-product-derived peptidomimetic inhibitor of the NS3-4A protease. It is believed to be stabilized into the enzyme's active site through a ketoamide. See, e.g., Lin et al., 2005, J Biol. Chem. Manuscript M506462200 (epublication); Summa, 2005, Curr Opin Investig Drugs. 6:831-7, the contents of which are hereby incorporated by reference in their entireties.

In certain embodiments, the second agent is a modulator of the HCV NS5B The RNA-dependent RNA polymerase (RdRp). Contained within the NS5B protein, RdRp synthesizes RNA using an RNA template. This biochemical activity is not present in mammalian cells.

One useful modulator of RdRp is NM283 (Valopicitabine; Idenix/Novartis). NM283, is an oral prodrug (valine ester) of NM107 (2′-C-methyl-cytidine) in phase II trials for the treatment or prevention of HCV infection. See, e.g., U.S. Patent Application Publication No. 20040077587, the contents of which are hereby incorporated by reference in their entirety.

Other useful modulators of RdRp include 7-deaza nucleoside analogs. For instance, 7-Deaza-2′-C-methyl-adenosine is a potent and selective inhibitor of hepatitis C virus replication with excellent pharmacokinetic properties. Olsen et al., 2004, Antimicrob. Agents Chemother. 48:3944-3953, the contents of which are hereby incorporated by reference in their entirety.

In further embodiments, the second agent is a non-nucleoside modulator of NS5B. At least three different classes of non-nucleoside inhibitors (NNI) of NS5B inhibitors are being evaluated in the clinic.

Useful non-nucleoside modulators of NS5B include JTK-003 and JTK-009. JTK-003 has been advanced to phase II. Useful non-nucleoside modulators of NS5B include the 6,5-fused heterocyclic compounds based on a benzimidazole or indole core. See, e.g., Hashimoto et al., International Publication NO. WO 00147883, the contents of which are hereby incorporated by reference in their entirety.

Further useful polymerase NNIs include R803 (Rigel) and HCV-371, HCV-086 and HCV-796 (ViroPharma/Wyeth). Additional useful NNIs include thiophene derivatives that are reversible allosteric inhibitors of the NS5B polymerase and bind to a site that is close to, but distinct from, the site occupied by benzimidazole-based inhibitors. See, e.g., Biswal, et al., 2005, J. Biol. Chem. 280, 18202-18210 (2005).

Further useful NNIs for the methods provided herein include benzothiadiazines, such as benzo-1,2,4-thiadiazines. Derivatives of benzo-1,2,4-thiadiazine have been shown to be highly selective inhibitors of the HCV RNA polymerase. Dhanak, et al., 2002, J. Biol. Chem. 277:38322-38327, the contents of which are hereby incorporated by reference in their entirety.

Further useful NNIs for the methods provided herein, and their mechanisms, are described in LaPlante et al., 2004, Angew Chem. Int. Ed. Engl. 43:4306-4311; Tomei et al., 2003, J. Virol. 77:13225-13231; Di Marco et al., 2005, J. Biol. Chem. 280:29765-70; Lu, H., WO 2005/000308; Chan et al., 2004, Bioorg. Med. Chem. Lett. 14:797-800; Chan et al., 2004, Bioorg. Med. Chem. Lett. 14:793-796; Wang et al., 2003, J. Biol. Chem. 278:9489-9495; Love, et al., 2003, J. Virol. 77:7575-7581; Gu et al., 2003, J. Biol. Chem. 278:16602-16607; Tomei et al., 2004, J. Virol. 78:938-946; and Nguyen et al., 2003, Antimicrob. Agents Chemother. 47:3525-3530.

In further embodiments, the second agent is an agent that is capable of interfering with HCV RNA such as small inhibitory RNA (siRNA) or a short hairpin RNA (shRNA) directed to an HCV polynucleotide. In tissue culture, siRNA and vector-encoded short hairpin RNA shRNA directed against the viral genome, effectively block the replication of HCV replicons. See, e.g., Randall et al., 2003, Proc. Natl. Acad. Sci. USA 100:235-240.

In further embodiments, the second agent is an agent that modulates the subject's immune response. For instance, in certain embodiments, the second agent can be a presently approved therapy for HCV infection such as an interferon (IFN), a pegylated IFN, an IFN plus ribavirin or a pegylated IFN plus ribavirin. In certain embodiments, interferons include IFNα, IFNα2a and IFNα2b, and particularly pegylated IFNα2a (PEGASYS®) or pegylated IFNα2b (PEG-INTRON®).

In further embodiments, the second agent is a modulator of a Toll-like receptor (TLR). It is believed that TLRs are targets for stimulating innate anti-viral response. Suitable TLRs include, bur are not limited to, TLR3, TLR7, TLR8 and TLR9. It is believed that toll-like receptors sense the presence of invading microorganisms such as bacteria, viruses and parasites. They are expressed by immune cells, including macrophages, monocytes, dendritic cells and B cells. Stimulation or activation of TLRs can initiate acute inflammatory responses by induction of antimicrobial genes and pro-inflammatory cytokines and chemokines.

In certain embodiments, the second agent is a polynucleotide comprising a CpG motif. Synthetic oligonucleotides containing unmethylated CpG motifs are potent agonists of TLR-9. Stimulation of dendritic cells with these oligonucleotides results in the production of tumour necrosis factor-alpha, interleukin-12 and IFN-alpha. TLR-9 ligands are also potent stimulators of B-cell proliferation and antibody secretion. One useful CpG-containing oligonucleotide is CPG-10101 (Actilon; Coley Pharmaceutical Group) which has been evaluated in the clinic.

Another useful modulator of a TLR is ANA975 (Anadys). ANA975 is believed to act through TLR-7, and is known to elicit a powerful anti-viral response via induction and the release of inflammatory cytokines such as IFN-alpha.

In other embodiments, the second agent is Celgosivir. Celgosivir is an alpha-glucosidase I inhibitor and acts through host-directed glycosylation. In preclinical studies, celgosivir has demonstrated strong synergy with IFNα plus ribavirin. See, e.g., Whitby et al., 2004, Antivir Chem. Chemother. 15(3):141-51. Celgosivir is currently being evaluated in a Phase II monotherapy study in chronic HCV patients in Canada.

Further immunomodulatory agents, and their mechanisms or targets, are described in Schetter & Vollmer, 2004, Curr. Opin. Drug Discov. Dev. 7:204-210; Takeda et al., 2003, Annu. Rev. Immunol. 21:335-376; Lee et al., 2003, Proc. Natl. Acad. Sci. USA 100:6646-6651; Hosmans et al., 2004, Hepatology 40 (Suppl. 1), 282A; and U.S. Pat. No. 6,924,271.

In certain embodiments, the compounds provided herein may be administered in combination with one or more anti-cancer agents. Anti-cancer agents for use in combination with the instant compounds include, but are not limited to, an antifolate, a 5-fluoropyrimidine (including 5-fluorouracil), a cytidine analogue such as β-L-1,3-dioxolanyl cytidine or β-L-1,3-dioxolanyl 5-fluorocytidine, antimetabolites (including purine antimetabolites, cytarabine, fudarabine, floxuridine, 6-mercaptopurine, methotrexate, and 6-thioguanine), hydroxyurea, mitotic inhibitors (including CPT-11, Etoposide (VP-21), taxol, and vinca alkaloids such as vincristine and vinblastine, an alkylating agent (including but not limited to busulfan, chlorambucil, cyclophosphamide, ifofamide, mechlorethamine, melphalan, and thiotepa), nonclassical alkylating agents, platinum containing compounds, bleomycin, an anti-tumor antibiotic, an anthracycline such as doxorubicin and dannomycin, an anthracenedione, topoisomerase II inhibitors, hormonal agents (including but not limited to corticosteroids (dexamethasone, prednisone, and methylprednisone), androgens such as fluoxymesterone and methyltestosterone, estrogens such as diethylstilbesterol, antiestrogens such as-tamoxifen, LHRH analogues such as leuprolide, antiandrogens such as flutamide, aminoglutethimide, megestrol acetate, and medroxyprogesterone), asparaginase, carmustine, lomustine, hexamethyl-melamine, dacarbazine, mitotane, streptozocin, cisplatin, carboplatin, levamasole, and leucovorin. The compounds of the present invention can also be used in combination with enzyme therapy agents and immune system modulators such as an interferon, interleukin, tumor necrosis factor, macrophage colony-stimulating factor and colony stimulating factor.

It should be understood that any suitable combination of the compounds provided herein with one or more of the above-mentioned compounds and optionally one or more further pharmacologically active substances are considered to be within the scope of the present disclosure. In another embodiment, the compound provided herein is administered prior to or subsequent to the one or more additional active ingredients.

It will be apparent to those skilled in the art that many modifications, both to materials and methods, may be practiced without departing from the scope of the invention.

Finally, it should be noted that there are alternative ways of implementing both the present invention. Accordingly, the present embodiments are to be considered as illustrative and not restrictive, and the invention is not to be limited to the details given herein, but may be modified within the scope and equivalents of the appended claims.

All publications and patents cited herein are incorporated by reference in their entirety.

The following examples are provided for illustrative purposes only and are not intended to limit the scope of the invention.

### EXAMPLES

### Example 1

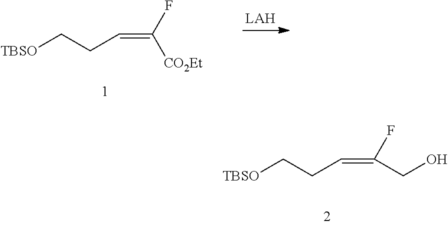 [SMILES: CCOC(=O)/C(F)=C\CCO[Si](C)(C)C(C)(C)C ; CC(C)(C)[Si](C)(C)OCC/C=C(/F)CO]

A solution of 1 (15.4 g, 0.0558 mol) which is known in the art, dissolved in THF was cooled to 0° C. and treated with lithium tetrahydroaluminate (2.3 g, 0.061 mol). This resulted in an exotherm to 440° C. The mixture slowly cooled to 0° C. and was allowed to stir for 15 min. 6 mL of water were slowly added along with Na₂SO₄. Stirring was continued for 45 min and then the gray mixture was filtered through a pad of Solka Floc®. Concentration afforded the product 2 as a clear oil in 76% yield. ¹H NMR (400 MHz, CHLOROFORM-d) δ ppm 0.76-0.88 (m, 26H); 1.13-1.26 (m, 5H); 1.71-1.81 (m, 1H); 2.14-2.27 (m, 5H); 3.52-3.59 (m, 5 H); 3.60-3.69 (m, 2H); 4.09 (br. s., 2H); 4.14 (br. s., 2H); 5.06-5.19 (m, 2H).

### Example 2

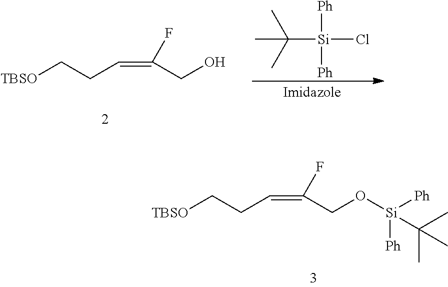 [SMILES: CC(C)(C)[Si](C)(C)OCC/C=C(/F)CO ; CC(C)(C)[Si](Cl)(c1ccccc1)c1ccccc1 ; CC(C)(C)[Si](C)(C)OCC/C=C(/F)CO[Si](c1ccccc1)(c1ccccc1)C(C)(C)C]

A solution of 2 (13.0 g, 0.0555 mol), 1H-imidazole (8.2 g, 0.12 mol) and tetrahydrofuran (100 mL, 1 mol) was cooled to 13° C. and t-butylchlorodiphenylsilane (15.7 mL, 0.0605 mol) was added so as to maintain the temperature between 13° C. and 16° C. When the addition was complete, the slurry was allowed to warm to room temperature and stirred for 1 hour. Water was added and the product extracted with hexane/ether. Concentration afforded 3 as a clear oil in 100% yield. ¹H NMR (400 MHz, CHLOROFORM-d) δ ppm 0.81-0.94 (m, 9H); 1.01-1.13 (m, 16H); 1.21-1.33 (m, 1H); 1.21-1.33 (m, 1H); 1.77-1.86 (m, 1H); 1.96-2.08 (m, 1H); 3.44-3.56 (m, 1H); 3.66-3.78 (m, 2H); 4.17-4.29 (m, 1H); 7.33-7.46 (m, 10H); 7.64-7.77 (m, 7H).

### Example 3

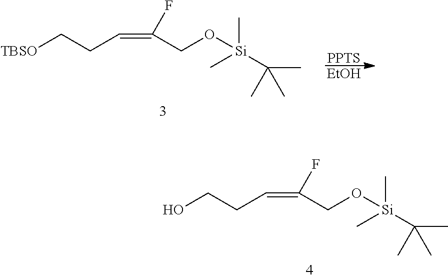

Pyridinium p-toluensulfonate 6 and ethanol (100 mL) were combined at room temperature and stirred overnight. The mixture was quenched with 10 mL of saturated bicarbonate, concentrated and filtered through Solka Floc® with ether to remove solids. The crude material was then kugelrohr distilled to remove the TBSOH and some TBDPSOH to provide 7 in 66% yield. ¹H NMR (400 MHz, CHLOROFORM-d) δ ppm 0.81-0.93 (m, 2H); 1.00-1.12 (m, 17H); 1.20-1.33 (m, 1H); 1.27 (s, 1H); 1.79 (br. s., 2H); 1.99-2.10 (m, 3H); 3.44-3.56 (m, 3H); 4.16-4.28 (m, 3H); 5.09-5.22 (m, 1H); 7.34-7.46 (m, 10H); 7.64-7.76 (m, 7H).

### Example 4

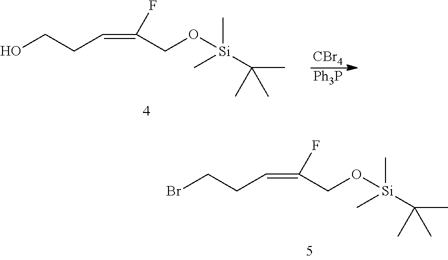

A solution of 4 (3E)-5-{[tert-butyl(diphenyl)silyl]oxy}-4-fluoropent-3-en-1-ol (12.7 g, 0.0354 mol) and carbon tetrabromide (12.9 g, 0.0390 mol) in 100 mL of CH₂Cl₂ at −58° C. was treated with a solution of triphenylphosphine (10.2 g, 0.0390 mol) in 50 mL of methylene chloride while maintaining the temperature between −55° C. and -58° C. The mixture was slowly warmed to room temperature and allowed to stir overnight. Hexane was added, the solution concentrated to remove the methylene chloride and then stirred at room temperature to crystallize out the triphenylphosphine. This was partially successful in that a few solids came out of solution. These were removed by filtration and then the mother liquor was concentrated to a thick oil. The product was purified by chromatography with 0-5% EtOAc/hex to provide 5 in 68% yield. ¹H NMR (400 MHz, CHLOROFORM-d) δ ppm 0.73-0.82 (m, 1H); 0.88-1.01 (m, 18H); 1.09-1.21 (m, 1H); 2.17-2.29 (m, 3H); 3.06-3.16 (m, 3H); 3.37-3.48 (m, 1H); 4.04-4.12 (m, 2H); 4.13-4.17 (m, 1H); 4.98-5.11 (m, 2H); 7.23-7.36 (m, 11H); 7.53-7.63 (m, 7H).

### Example 5

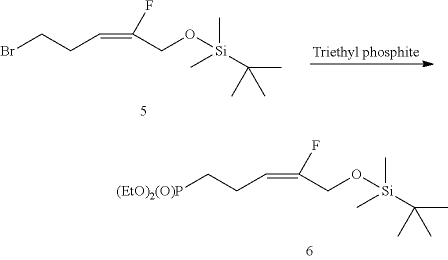

The bromide 5 was dissolved in 100 mL of triethyl phosphite (100 mL, 0.6 mol) and heated at 155° C. overnight. Excess phosphite was then removed by vacuum distillation at 70° C. The residue was chromatographed on about 200 g of silica with 50-70% EtOAc/hex to provide 6 in 70% yield. ¹H NMR (400 MHz, CHLOROFORM-d) δ ppm 0.89-1.01 (m, 7H); 0.95 (s, 7 H); 1.13-1.24 (m, 3H); 1.17 (t, J=7.07 Hz, 6H); 1.54-1.66 (m, 2H); 1.93 (s, 1H); 1.98-2.10 (m, 2H); 3.90-4.02 (m, 6H); 4.08-4.20 (m, 2H); 5.00-5.12 (m, 1H); 7.24-7.36 (m, 8H); 7.52-7.64 (m, 5H).

### Example 6

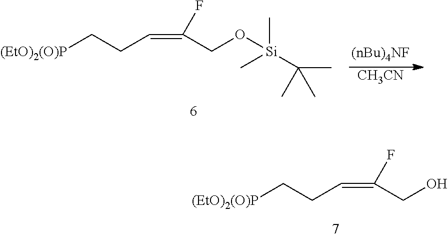

Diethyl[(3E)-5-{[tert-butyl(diphenyl)silyl]oxy}-4-fluoropent-3-en-1-yl]phosphonate 7 (7.7 g, 0.016 mol) in acetonitrile was treated with tetrabutylammonium fluoride (4.72 g, 0.0169 mol) at 0° C. and allowed to stir for 1 hour. The mixture was concentrated and then chromatographed on silica with 5% MeOH/CH₂Cl₂ to give the allylic alcohol 7 in 76% yield. ¹H NMR (400 MHz, CHLOROFORM-d) δ ppm 1.21-1.34 (m, 18H); 1.77-1.88 (m, 6H); 2.29-2.41 (m, 6H); 3.99-4.09 (m, 12H, 4.11-4.13 (m, 3H); 4.15-4.20 (m, 3H); 5.06-5.18 (m, 3H).

### Example 7

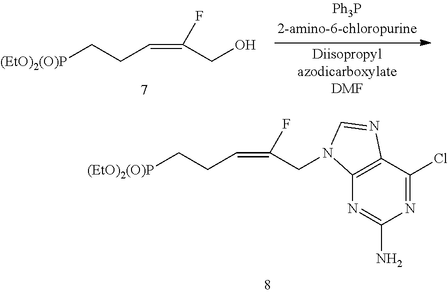

Diethyl[(3E)-4-fluoro-5-hydroxypent-3-en-1-yl]phosphonate 7 (1.046 g, 0.004354 mol), 2-amino-6-chloropurine (0.738 g, 0.00435 mol) and triphenylphosphine (1.14 g, 0.00435 mol) was dissolved in DMF, cooled to 0° C. and slowly treated with diisopropyl azodicarboxylate (0.9025 mL, 0.004354 mol) so as to keep the temperature at less than 4° C. When the addition was complete the reaction mixture was stirred overnight at room temperature. The solvent was removed by kugelrohr distillation at high vacuum at 45° C. The crude oil which contained the product by MS was then chromatographed on silica gel with 2.5-10% MeOH/methylene chloride. The product came off in the 5% MeOH fractions. The fraction containing the product crystallized but still contained some triphenylphosphine oxide. The solids were triturated with EtOAc to afford 800 mg of desired product (50% yield). The mother liquor was concentrated and an additional amount of solid isolated (112 mg, 6.8%). By LCMS this contained a little Ph₃P. The second isomer was crystallized from EtOAc to give some pale yellow solid. The ¹HNMR looks similar to desired except that the big doublet is substantially down field. ¹³C NMR (101 MHz, CHLOROFORM-d) δ ppm 16.47 (s, 1C); 16.53 (s, 1C); 19.22 (s, 1C); 19.26 (s, 1C); 19.31 (s, 1C); 19.34 (s, 1C); 25.33 (s, 1C); 26.72 (s, 1C); 40.10 (s, 1C); 40.41 (s, 1C); 61.89 (s, 1C); 61.96 (s, 1C); 76.73 (s, 1C); 77.05 (s, 1C); 77.15 (s, 1C); 77.36 (s, 1C); 77.47 (s, 1C); 110.64 (s, 1C); 110.85 (s, 1C); 141.71 (s, 1C); 153.63 (s, 1C); 159.85 (s, 1C). ¹H NMR (400 MHz, CHLOROFORM-d) δ ppm 1.32-1.44 (m, 15H); 1.84-1.96 (m, 5H); 2.67-2.79 (m, 5H); 4.09-4.21 (m, 10H); 4.76-4.81 (m, 2H); 4.81-4.94 (m, 3H); 5.28 (dt, J=19.23, 8.06 Hz, 3H); 6.07 (br. s., 4H); 7.78-7.89 (m, 3H).

### Example 8

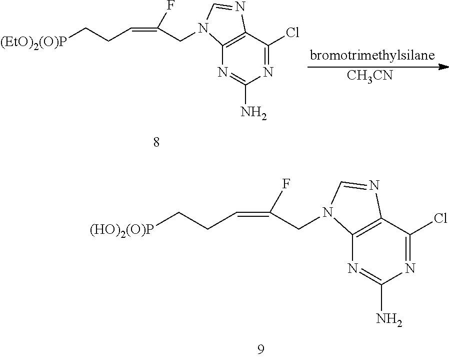

Diethyl[(3E)-5-(2-amino-6-chloro-9H-purin-9-yl)-4-fluoropent-3-en-1-yl]phosphonate 11 (800 mg, 0.00204 mol) in acetonitrile (2.0 mL, 0.038 mol) was treated with bromotrimethylsilane (0.620 mL, 0.00470 mol) and allowed to stir at room temperature overnight. The reaction mixture was quenched with saturated NaHCO₃ and the pH adjusted to 8.5. The clear solution was loaded onto a Dowex 1×2 resin and washed with 100 mL of water. The product was eluted with a gradient of 1-5% formic acid in MeOH. The crude material was concentrated and crystallized from MeOH to provide 9 in 96% yield. ¹H NMR (400 MHz, DMSO-d6) δ ppm 1.73 (s, 14H); 3.13-3.22 (m, 10H); 4.84-4.92 (m, 7H); 4.95 (s, 7H); 5.38 (s, 3H); 5.44 (s, 3H); 8.13-8.23 (m, 7H).

### Example 9

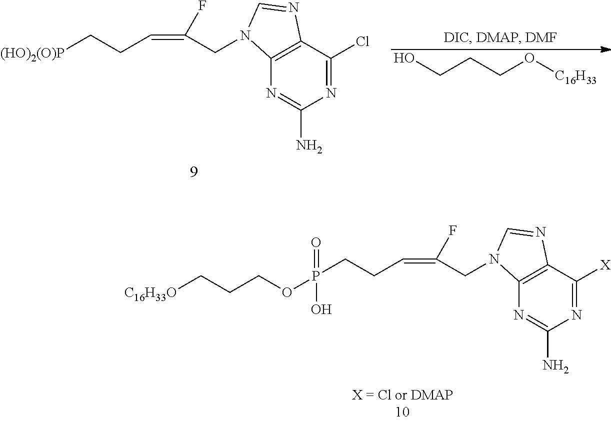

[(3E)-5-(2-amino-6-chloro-9H-purin-9-yl)-4-fluoropent-3-en-1-yl]phosphonic acid 9 (596 mg, 0.00178 mol), 3-(hexadecyloxy)propan-1-ol (0.755 g, 0.00251 mol) and 4-dimethylaminopyridine (0.31 g, 0.0025 mol) in DMF at room temperature was treated with N,N′-diisopropylcarbodiimide (0.814 mL, 0.00520 mol) and the mixture heated at 50° C. for 1.4 hours. The reaction mixture was then warmed to 70° C. and stirred overnight. Water was then added and an attempt was made to extract the excess alcohol with MTBE. This failed in that a homogeneous soapy solution resulted. The solvent was then evaporated and the residue chromatographed on 100 mL of silica with the following solvent: 900 mL:100 mL:15 mL: CHCl₃:MeOH:conc NH₄OH DMAP to afford 10 (X is Cl) in 20% yield and 10 (X is DMAP) in 24% yield. ¹H NMR (400 MHz, MeOD) δ ppm 0.86-0.97 (m, 3H); 1.23-1.36 (m, 23H); 1.49 (br. s., 2H); 1.73 (s, 2H); 1.88 (t, J=6.32 Hz, 2H); 3.30-3.35 (m, 3H); 3.39 (t, J=6.66 Hz, 2 H); 3.55 (t, J=6.35 Hz, 2H); 3.97 (q, J=6.24 Hz, 2H); 4.91 (s, 6H) 4.98 (s, 1H); 5.03 (s, 1H) 8.09-8.14 (m, 1H). ¹H NMR (400 MHz, MeOD) δ ppm 0.84-0.97 (m, 2H); 1.22-1.34 (m, 8 H); 1.22-1.34 (m, 13H); 1.46-1.56 (m, 2H); 1.70-1.82 (m, 2H); 1.91 (quin, J=6.32 Hz, 2H); 2.57-2.70 (m, 1H); 3.36-3.48 (m, 2H); 3.43 (s, 4H); 3.56 (t, J=6.35 Hz, 2H); 4.01 (q, J=6.34 Hz, 2H); 4.93 (s, 4H); 4.99 (s, 1H); 5.04 (s, 1H); 5.36-5.48 (m, 1H); 7.12-7.25 (m, 2H); 8.03-8.16 (m, 1H); 9.47-9.59 (m, 1H).

### Example 10

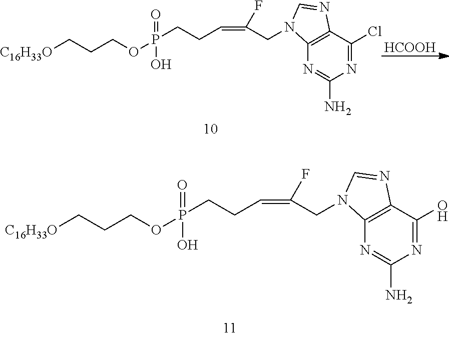

3-(hexadecyloxy)propyl hydrogen[(3E)-5-(2-amino-6-chloro-9H-purin-9-yl)-4-fluoropent-3-en-1-yl]phosphonate 10 (184 mg, 0.000298 mol) in formic acid (4 mL, 0.1 mol) was heated at 95° C. After 45 minutes the reaction was about 40% complete. After about 2 hours the reaction was complete by LCMS. The solvent was removed to afford a white solid. The solid was triturated with MeOH to afford the desired product by NMR. The product and mother liquor were recombined in MeOH and partially concentrated. This resulted in slow precipitation of the product. The mixture was cooled in an ice bath for 1 hour and then the MeOH was removed with a pipet. The solid was dried under vacuum to provide 11 in 785 yield. ¹H NMR (400 MHz, DMSO-d6) δ ppm 0.80-0.92 (m, 3H); 1.18-1.30 (m, 24H); 1.77 (t, J=6.32 Hz, 3H); 2.51 (dt, J=3.77, 1.87 Hz, 8H); 3.31 (t, J=6.58 Hz, 3H); 3.40 (t, J=6.30 Hz, 3H); 3.89 (d, J=6.89 Hz, 2 H); 4.75 (s, 1H) 4.81 (s, 1H); 7.67 (s, 1H).

### Example 11

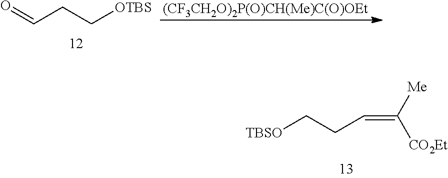 [SMILES: CC(C)(C)[Si](C)(C)OCCC=O ; CCOC(=O)/C(C)=C\CCO[Si](C)(C)C(C)(C)C ; "(CF3CH2O)2P(O)CH(Me)C(O)OEt"]

A 1 L flask was charged with tetrahydrofuran (500 mL, 6 mol), potassium bis(trimethylsilyl)amide (12.9 g, 0.0647 mol) and 1,4,7,10,13,16-hexaoxacyclooctadecane (0.181 mol). Ethyl 2-[bis(2,2,2-trifluoroethoxy)phosphoryl]propanoate (28.0 g, 0.0647 mol) was then added after cooling to −78° C. The aldehyde 3, which is known in the art, in 20 mL of THF was then added and the mixture slowly allowed to warm to 15° C. overnight. The reaction mixture was then quenched with NH₄Cl solution and the product extracted with MTBE. The extract was washed with saturated K₂CO₃ and concentrated. The concentrate was taken up in MTBE and washed 2× with water. The organic layer was concentrated to give more than 100% yield of 13. ¹H NMR (400 MHz, CHLOROFORM-d) δ ppm 0.78-0.91 (m, 23H) 1.20-1.32 (m, 6H) 1.42-1.51 (m, 1H) 1.66 (t, J=6.19 Hz, 1H) 1.79-1.91 (m, 5H) 2.57-2.68 (m, 2H) 3.59-3.70 (m, 5 H) 4.09-4.21 (m, 4H) 4.31-4.44 (m, 2H) 5.91-5.99 (m, 1H).

### Example 12

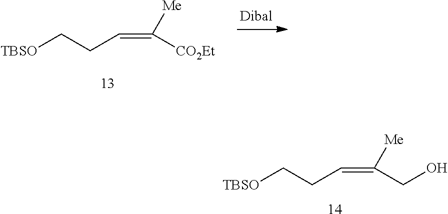

The crude ethyl (2Z)-5-{[tert-butyl(dimethyl)silyl]oxy}-2-methylpent-2-enoate (17.6 g, 0.0647 mol) 13 was dissolved in 50 mL of THF and slowly add to 200 mL of a 1M DIBAL solution in 200 mL of methylene chloride at −72° C. The mixture was allowed to stir for 2.5 hours and quenched with methanol followed by Rochelle's salt solution in water which eventually broke the usual emulsion after standing in separatory funnel overnight. The layers were separated and the organic dried over sodium sulfate and concentrated to provide 14 in 91% yield. ¹H NMR (400 MHz, CHLOROFORM-d) δ ppm 0.77-0.89 (m, 29H); 1.16-1.24 (m, 1 H); 1.61-1.68 (m, 1H); 1.70-1.81 (m, 5H); 2.20-2.32 (m, 3H); 2.54-2.66 (m, 1H); 3.56 (t, J=6.06 Hz, 2H); 3.60-3.69 (m, 3H); 3.97-4.08 (m, 1H); 3.99 (s, 2H); 5.20-5.30 (m, 2H).

### Example 13

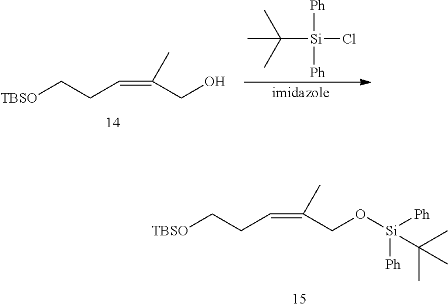

The crude alcohol 14 was dissolved in 100 mL of THF, treated with imidazole and cooled to 11° C. A solution of the silyl chloride in 40 mL of THF was then slowly added keeping the temperature between 10° C. and 12° C. After 3 hours the mixture was quenched with water and the product 15 was isolated with MTBE in 99% yield. The crude was used as is in the subsequent step.

### Example 14

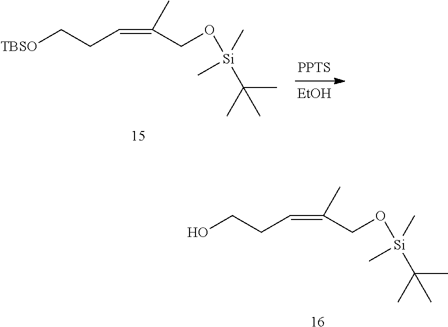

(6Z)-2,2,6,11,11,12,12-heptamethyl-3,3-diphenyl-4,10-dioxa-3,11-disilamidec-6-ene (30.3 g, 0.0646 mol) 15 and pyridinium p-toluenesulfonate (1.32 g, 0.00525 mol) in ethanol (190 mL, 3.2 mol) were stirred at room temperature for 24 hours. The reaction was quenched with saturated bicarbonate and partially concentrated. The oil was taken up in MTBE and water and the phases separated. The MTBE layer was dried over MgSO₄, filtered and concentrated. Low boiling impurities were removed by kugelrohr distillation under high vacuum at 70° C. NMR showed that the hydrolysis was not complete and as a result the mixture was resubjected to the reaction conditions for a second overnight run. This material was then filtered through a plug of magnesol with MTBE and concentrated to provide 18 in about 92% yield. Even after high vacuum overnight it contained about 12% MTBE. ¹H NMR (400 MHz, CHLOROFORM-d) δ ppm 0.74-0.86 (m, 1H); 0.92-1.05 (m, 16H); 1.07-1.16 (m, 3H); 1.66-1.79 (m, 5H); 2.01 (m, J=7.61, 6.41, 6.41, 1.14 Hz, 2H); 3.05-3.14 (m, 1H); 3.34-3.46 (m, 2H); 4.02-4.13 (m, 2H); 5.07-5.19 (m, 1H); 7.24-7.37 (m, 10H); 7.55-7.67 (m, 7H).

### Example 15

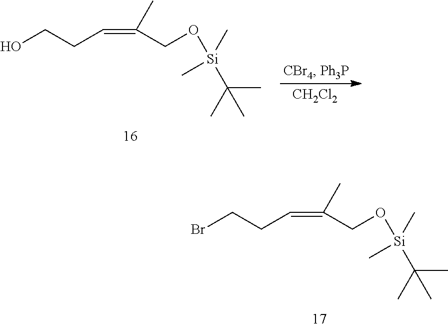

(3Z)-5-{[tert-butyl(diphenyl)silyl]oxy}-4-methylpent-3-en-1-ol (22.12 g, 0.06239 mol) 16 and carbon tetrabromide (24.2 g, 0.0730 mol) in 200 mL of methylene chloride was cooled to −78° C. and treated with a solution of triphenylphosphine (19.6 g, 0.0749 mol) in 80 mL of methylene chloride while keeping the temperature at <−70° C. The mixture was slowly allowed to warm to room temperature overnight. The crude mixture was concentrated and chromatographed with 1-2% EtOAc/Hex to provide bromide 17 in 53% yield. ¹H NMR (400 MHz, CHLOROFORM-d) δ ppm 0.91-1.03 (m, 16H); 1.72-1.82 (m, 5H); 2.21-2.33 (m, 4H); 3.10 (t, J=7.18 Hz, 3H); 4.03-4.15 (m, 4H); 5.04-5.16 (m, 2H); 7.25-7.37 (m, 10H); 7.55-7.67 (m, 7H).

### Example 16

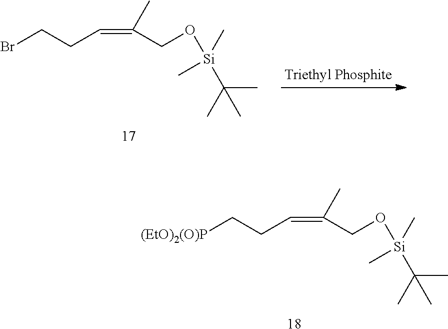

{[(2Z)-5-bromo-2-methylpent-2-en-1-yl]oxy}(tert-butyl)diphenylsilane (9.20 g, 0.0220 mol) 17 was dissolved in triethyl phosphite (30 mL, 0.2 mol) and heated at 150° C. for 26 hours. The excess phosphite was removed by distillation to provide 18 in about 100% yield. ¹H NMR (400 MHz, CHLOROFORM-d) δ ppm 1.01-1.13 (m, 12H); 1.21-1.28 (m, 7H); 1.30-1.35 (m, 2H); 1.61-1.72 (m, 2H); 1.77-1.89 (m, 4H); 2.08-2.21 (m, 2H); 3.98-4.06 (m, 4H); 4.08-4.14 (m, 1H); 4.17-4.22 (m, 2H); 5.15-5.26 (m, 1H); 7.34-7.47 (m, 8H); 7.64-7.76 (m, 5H).

### Example 17

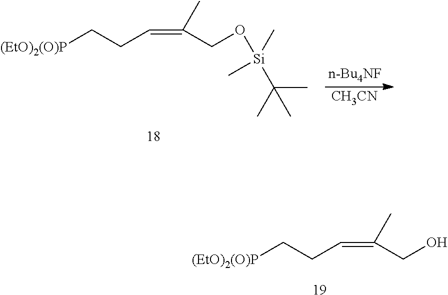

A solution of the crude diethyl[(3Z)-5-{[tert-butyl(diphenyl)silyl]oxy}-4-methylpent-3-en-1-yl]phosphonate 18 (10.4 g, 0.0220 mol) in acetonitrile at 0° C. was treated with n-tetrabutylammonium fluoride hydrate (6.8 g, 0.024 mol) and stirred for 5 h when TLC shows the reaction to be complete. The mixture was filtered through a plug of silica gel to remove the n-Bu₄N and then chromatographed on 150 g of silica with 10-15% IPA/EtOAc to provide the alcohol 19 in 76% yield. ¹H NMR (400 MHz, CHLOROFORM-d) δ ppm 1.06-1.17 (m, 2H); 1.20-1.32 (m, 15H); 1.72-1.83 (m, 12H); 2.29-2.42 (m, 5H); 3.95-4.08; (m, 14H) 5.14-5.27 (m, 2H).

### Example 18

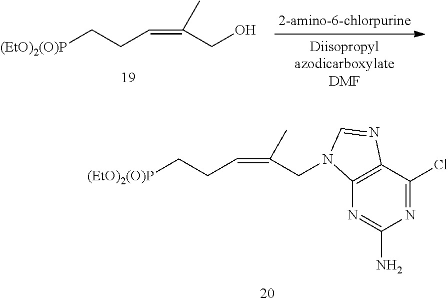

Diethyl[(3Z)-5-hydroxy-4-methylpent-3-en-1-yl]phosphonate (1.50 g, 0.00635 mol) 19 and 2-amino-6-chloropurine (1.23 g, 0.00724 mol) were suspended in N,N-dimethylformamide (20.0 mL, 0.258 mol) and treated with triphenylphosphine (1.90 g, 0.00724 mol). The slurry was cooled to 0° C. and then slowly treated with diisopropyl azodicarboxylate (1.42 mL, 0.00724 mol) in an exothermic reaction. The solution was slowly allowed to warm to room temperature and stir overnight. The mixture was concentrated and chromatographed on silica gel with 5-10% MeOH/methylene chloride to provide 20 in 51% yield and its regiosiomer in about 15% yield. Both regioisomers were isolated but the undesired isomer one was not totally pure. ¹H NMR of depicted regioisomer 20 (400 MHz, CHLOROFORM-d) δ ppm 1.29 (d, J=6.22 Hz, 2H); 1.36 (t, J=7.07 Hz, 11H); 1.65 (s, 6H); 1.67-1.75 (m, 6H); 1.92 (dd, J=17.91, 16.46 Hz, 4H); 4.08-4.21 (m, 7H); 4.61-4.69 (m, 4H); 7.28 (s, 3H); 7.76 (s, 3H). ¹H NMR of incorrect regioisomer (400 MHz, CHLOROFORM-d) δ ppm 1.18-1.21 (m, 3H); 1.23-1.35 (m, 11H); 1.56-1.60 (m, 5H); 1.81-1.92 (m, 4H); 2.43-2.55 (m, 3H); 3.41-3.50 (m, 4H); 4.05-4.15 (m, 7H); 4.94-5.05 (m, 4H); 5.27-5.35 (m, 4H); 5.50-5.59 (m, 2H); 7.73-7.84 (m, 1H).

### Example 19

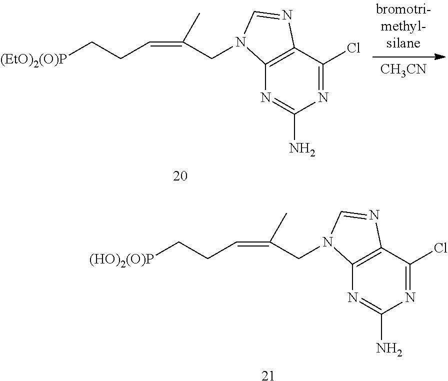

The phosphonate 20 (1 g, 0.00258 mole), bromotrimethylsilane (0.85 mL, 0.0064 mole) and solvent (5 mL) were combined and stirred for 4 hours. The reaction was quenched with saturated NaHCO₃ and the pH adjusted to 8.5. The product was loaded onto a Dowex 1×2 resin and eluted with 1-10% formic acid/MeOH to give the very insoluble solid 21 in 76% yield. ¹H NMR (400 MHz, DMSO-d6) δ ppm 2.51 (dt, J=3.73, 1.87 Hz, 20H); 8.14 (s, 2H); 8.11 (s, 3H).

### Example 20

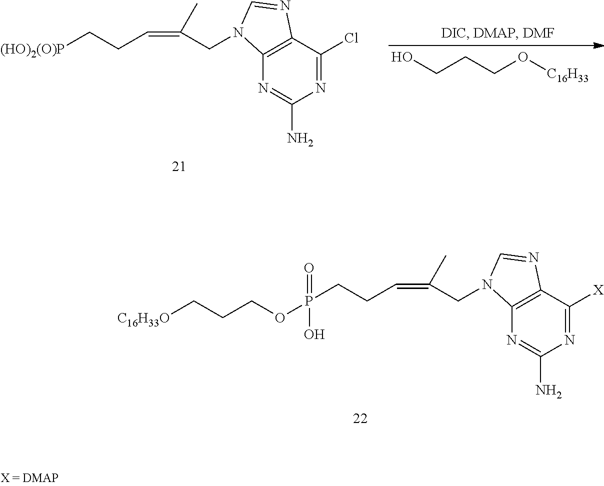

[(3E)-5-(2-amino-6-chloro-9H-purin-9-yl)-4-methylpent-3-en-1-yl]phosphonic acid 221 (650 mg, 0.0020 mol), 3-(hexadecyloxy)propan-1-ol (0.833 g, 0.00294 mol), 4-dimethylaminopyridine (0.31 g, 0.0025 mol) in DMF (5.0 mL) at room temperature were treated with N,N′-diisopropylcarbodiimide (0.890 mL, 0.00568 mol) and the mixture was heated at 65° C. overnight. The solvent was removed and the entire mixture was loaded onto silica gel and chromatographed with 1000:200:10 CHCl₃:MeOH:ammonia. The major product was the DMAP adduct 22. ¹H NMR (400 MHz, MeOD) δ ppm 0.86-0.97 (m, 2H); 1.06-1.17 (m, 2H); 1.12 (d, J=6.53 Hz, 3H); 1.19-1.30 (m, 14H); 1.30-1.40 (m, 2H); 1.50 (br. s., 1H); 1.57-1.69 (m, 2H); 1.73 (s, 1H); 1.89 (t, J=6.30 Hz, 1H); 3.30-3.34 (m, 2H); 3.36-3.46 (m, 2H); 3.43 (s, 3 H); 3.56 (t, J=6.32 Hz, 1H); 3.99 (d, J=6.17 Hz, 1H); 4.82-4.93 (m, 5H); 4.90 (s, 5H); 7.19-7.28 (m, 1H); 8.18 (s, 1H); 9.61-9.70 (m, 1H).

### Example 21

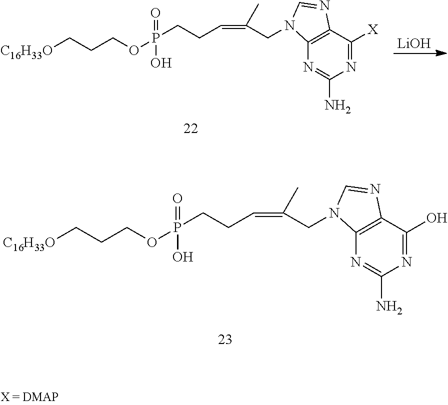

3-(hexadecyloxy)propyl[(3Z)-5-{2-amino-6-[4-(dimethylamino)pyridinium-1-yl]-9H-purin-9-yl}-4-methylpent-3-en-1-yl]phosphonate 22 (398 mg, 0.000509 mg), LiOH 90.12 g, 0028 mol), THF (5 mL) and water 92 mL) were combined and stirred at room temperature for ½ hour. LCMS shows the formation of product but slowly. The mixture was then heat at 50° C. until LCMS showed the disappearance of starting material. The mixture was acidified with formic acid and concentrated. The crude material was chromatographed on silica with 500 mL:50 mL:5 mL CHCl₃: MeOH: ammonia then 400:100:10, then 300 200 20 until the desired product eluted. Formic acid was added, the solution concentrated and the residue was crystallized from MeOH at −10° C. to afford 23 as an off white solid. ¹H NMR (400 MHz, DMSO-d6) δ ppm 0.85 (s, 3H) 1.22; (d, J=4.92 Hz, 23H); 1.53 (s, 4H); 2.51 (dt, J=3.73, 1.87 Hz, 9H); 2.52 (br. s., 2H); 3.30 (s, 3H) 3.39 (s, 2H) 7.61-7.65 (m, 1H).

### Example 22

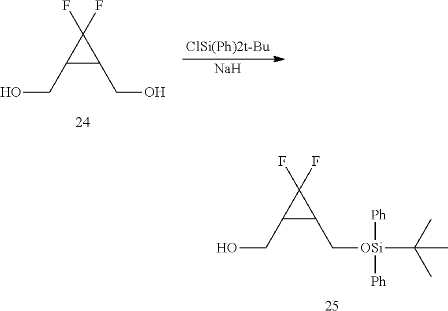

Sodium hydride (1.41 g, 0.0353 mol) in tetrahydrofuran (50 mL, 0.6 mol) was treated with the diol 24 (4.78 g, 0.0346 mol) (Itoh et al., J. Fluorine Chem. 2004, 125, 775-783) (4.00 g, 0.0106 mol) at 0° C. After 20 minutes tert-butylchlorodiphenylsilane (9.0 mL, 0.035 mol) was added and the mixture allowed to stir for 1 hour. Water was added and the product extracted with MTBE to afford an oil which was chromatographed on silica with 10% EtOAc/Hexane to provide 25 in 59% yield. ¹H NMR (400 MHz, CHLOROFORM-d) δ ppm 0.84-0.96 (m, 1H); 1.04-1.15 (m, 7H); 1.09 (s, 12H); 1.24-1.37 (m, 2H); 1.98 (m, J=13.66, 11.08, 11.08, 6.22 Hz, 2H); 2.14-2.26 (m, 2H); 3.14 (br. s., 2H); 3.76-3.85 (m, 3H); 3.92-4.05 (m, 3H); 7.40-7.52 (m, 10H); 7.64-7.77 (m, 7H).

### Example 23

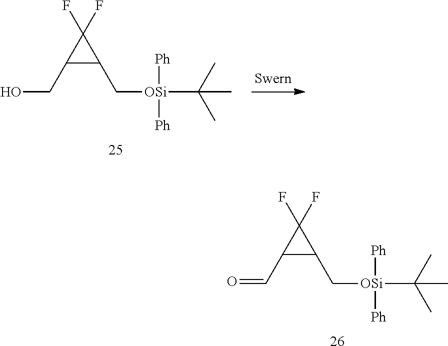

Oxalyl chloride (1.26 mL, 0.0149 mol) in 60 mL of methylene chloride was cooled to −70° C. and slowly treated with a solution of dimethyl sulfoxide (1.66 mL, (0.0234 mol) in 20 mL of methylene chloride keeping the temperature below −67° C. When the addition was complete the solution was stirred 10 min and then a solution of [3-({[tert-butyl(diphenyl)silyl]oxy}methyl)-2,2-difluorocyclopropyl]methanol 25 in 20 mL of methylene chloride was slowly added keeping the temperature below −68° C. The slurry was stirred for 30 minutes and then triethylamine (7.4 mL, 0.053 mol) was slowly added while keeping the temperature below −65° C. The mixture was stirred for 1 hour, warmed to 0° C., water was added and the mixture was then extracted with methylene chloride. The methylene chloride solution was dried over MgSO₄, filtered and concentrated to provide 26 as a clear oil in 100% yield. ¹H NMR (400 MHz, CHLOROFORM-d) δ ppm 0.87-0.97 (m, 2H); 1.00-1.12 (m, 18H); 1.26-1.38 (m, 2H); 2.36-2.48 (m, 2H); 2.53-2.66 (m, 3H); 3.99-4.12 (m, 2H); 4.13-4.24 (m, 2H); 7.40-7.52 (m, 11H); 7.63-7.73 (m, 7H); 9.32-9.43 (m, 2H).

### Example 24

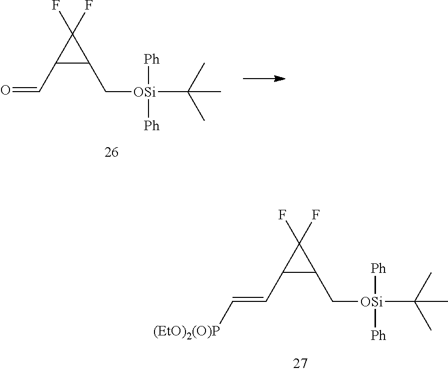

Sodium hydride (474 mg, 0.0118 mol) in tetrahydrofuran (40.0 mL, 0.493 mol) was cooled to −50° C. and treated with a THF (5 mL) solution of tetraethyl dimethylaminomethylene diphosphona (3.38 mL, 0.0118 mol). When the reaction became clear and gas evolution ceased a solution of 3-({[tert-butyl(diphenyl)silyl]oxy}methyl)-2,2-difluorocyclopropanecarbaldehyde 26 (3.70 g, 0.00988 mol) in 5 mL of THF was slowly added keeping the temperature below −50° C. The reaction mixture was then cooled to −78° C. and slowly allowed to warm to room temperature overnight. The reaction mixture was then poured into water and extracted with EtOAc, dried over MgSO₄, concentrated, dissolved in MTBE and filtered through a plug of Magnesol and concentrated to provide 27 in 100% yield. ¹H NMR (400 MHz, CHLOROFORM-d) δ ppm 0.84-0.96 (m, 1H); 1.01-1.13 (m, 12H); 1.19-1.22 (m, 4H); 1.21 (s, 4H); 1.25-1.38 (m, 8H); 2.17 (s, 1H); 3.24 (s, 1H); 3.81-3.86 (m, 1H); 3.88-3.92 (m, 1H); 3.99-4.12 (m, 4H); 5.87-5.99 (m, 1H); 7.37-7.49 (m, 8H); 7.63-7.70 (m, 5H).

### Example 25

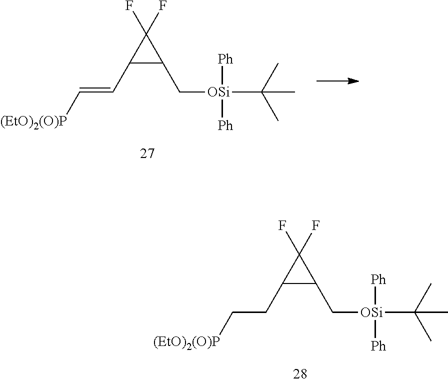

Diethyl {(E)-2-[3-({[tert-butyl(diphenyl)silyl]oxy}methyl)-2,2-difluorocyclopropyl]vinyl}phosphonate (4.40 g, 0.00865 mol) 27 in ethanol (30 mL, 0.5 mol) was treated with 10% palladium on carbon (0.50 g, 0.00047 mol) and hydrogenated at 13 psi for 4 hours and then 20 psi for another 4 hours to provide after concentration 28 in 100% yield. ¹H NMR (400 MHz, CHLOROFORM-d) δ ppm 1.01-1.13 (m, 13H); 1.26-1.38 (m, 10H); 1.79-1.90 (m, 3H); 3.70-3.80 (m, 2H); 3.80-3.92 (m, 2H); 4.02-4.15 (m, 5H); 7.37-7.49 (m, 9 H); 7.66-7.78 (m, 6H).

### Example 26

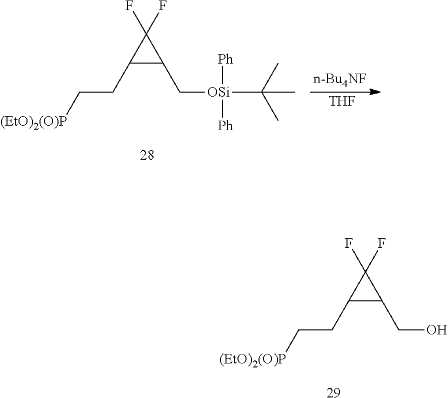

Diethyl {2-[(1R,3R)-3-({[tert-butyl(diphenyl)silyl]oxy}methyl)-2,2-difluorocyclopropyl]ethyl}phosphonate 28 (4.77 g, 0.00934 mol), N-Bu₄NF (2.87 g 0.0103 mol) and THF (5 mL) were combined at 0° C. and then allowed to warm up by removing the ice bath. When TLC showed the reaction to be complete the mixture was concentrated and chromatographed on silica with 5% MeOH/CH₂Cl₂. 1H NMR (400 MHz, CHLOROFORM-d) ppm 1.32 (t, 6H) 1.55-2.21 (m, 6H) 3.56-3.94 (m, 3H) 3.96-4.22 (m, 4H).

### Example 27

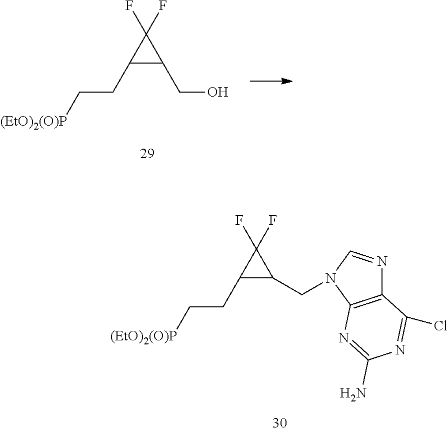

Diethyl {2-[2,2-difluoro-3-(hydroxymethyl)cyclopropyl]ethyl}phosphonate 29 (1.624 g, 0.005966 mol), 2-amino-6-chloropurine (1.01 g, 0.00596 mol), triphenylphosphine (1.56 g, 0.00596 mol) were dissolved in DMF, cooled to 0° C. and slowly treated with diisopropyl azodicarboxylate (1.236 mL, 0.005966 mol) while keeping the temperature at less than −7° C. When the addition was complete the reaction mixture was stirred overnight at room temperature. The next day the solvent was removed by kugelrohr distillation at high vacuum at 45° C. The crude oil which contains the product by MS was then chromatographed on silica gel (2×) with 5% MeOH/methylene chloride to provide 30 in 59% yield. MS (ESI−) for C₁₅H₂₁ClF₂N₅O₃P m/z 422.0 (M−H)⁻.

### Example 28

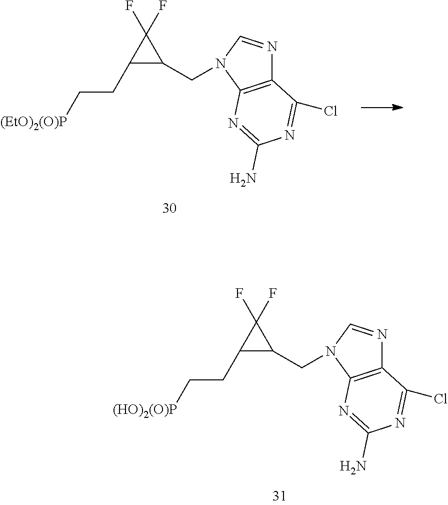

A solution of diethyl (2-{3-[(2-amino-6-chloro-9H-purin-9-yl)methyl]-2,2-difluorocyclopropyl}ethyl)phosphonate, 30 (1.44 g, 0.00340 mol) and bromotrimethylsilane (1.34 mL, 0.0102 mol) in acetonitrile (15 mL) were stirred at room temperature overnight. The resulting slurry was treated with an additional 0.6 mL of bromotrimethylsilane. The mixture was quenched with saturated sodium bicarbonate and the pH adjusted to 8.5 and purified using Dowex resin, eluting with 1-5% formic acid in methanol. MS (ESI−) for C₁₁H₁₃ClF₂N₅O₃P m/z 366.0 (M−H)⁻.

### Example 29

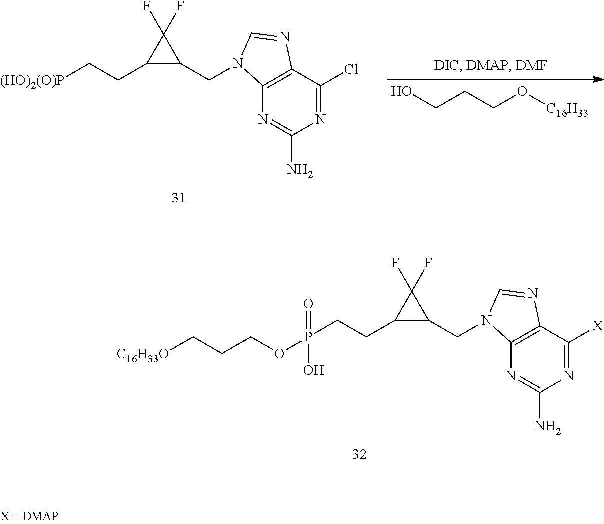

(2-{(1S,3S)-3-[(2-amino-6-chloro-9H-purin-9-yl)methyl]-2,2-difluorocyclopropyl}ethyl)phosphonic acid 31, (839 mg, 0.0022 mol) 3-(hexadecyloxy)propan-1-ol (1.02 g, 0.00339 mol), N,N′-diisopropylcarbodiimide (1.02 mL, 0.00655 mol), 4-dimethylaminopyridine 0.35 g, 0,0028 mol) and N,N-dimethylformamide (5.8 mL) were combined at room temperature and heated at 65° C. overnight. The next day the DMF was removed by concentration and the entire mixture loaded onto silica gel and chromatographed with 1000:200:10 CHCl₃:MeOH:Ammonia, 1000:300:20, then 600:400:40. The major product was the DMAP adduct 32 in 18% yield. MS (ESI+) for C₃₇H₆₀F₂N₇O₄P m/z 736.4 (M+H)⁺.

### Example 30

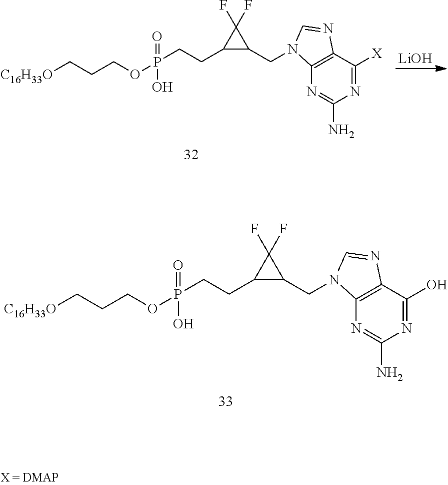

3-
(hexadecyloxy)propyl{2-[(1S,3S)-3-({2-amino-6-[4-(dimethylamino)pyridinium-1-yl]-9H-purin-9-yl}methyl)-2,2-difluorocyclopropyl]ethyl}phosphonate 32 929 mg 0.000404 mol), LiOH (0.1 g, 0.00236 mol), THF (5 mL) and water (2 mL) were combined and stirred at room temperature for 0.5 hour and then heated at 50° C. until the starting material disappeared. The mixture was acidified with formic acid and concentrated. The crude residue was chromatographed on silica with CHCl₃:MeOH:ammonia 400:100:10 then 300:200:20. The fractions were pooled and the product concentrated. The material was dissolved in MeOH and treated with formic acid and concentrated to convert it to the acid form. The product was then crystallized from cold MeOH and dried under high vacuum for 5 days to remove the water to provide 33 in 47% yield. MS (ESI+) for C₃₀H₅₂F₂N₅O₅P m/z 632.2 (M+H)⁺.

### Example 31

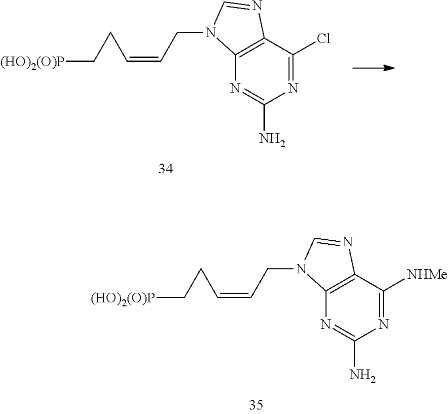

[(3Z)-5-(2-amino-6-chloro-9H-purin-9-yl)pent-3-en-1-yl]phosphonic acid 34 (Choo et al., Bioorg. Med. Chem 2007, 15, 2007) (0.509 g, 0.00160 mol) was treated with 2 M of methylamine in methanol (9.0 mL) at room temperature and stirred at room temperature overnight. MS (ESI+) for C₁₁H₁₇N₆O₃P m/z 312.9 (M+H)⁺.

### Example 32

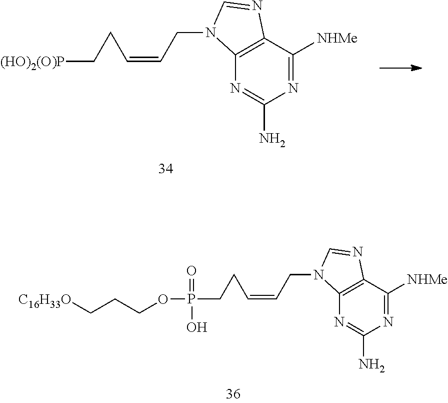

The viscous oil from Example 31 was taken up in 9.0 mL of DMF and DMAP (0.401 g, 0.00328 mol) was added. The slurry was partially concentrated to remove the excess methylamine and then 3-(hexadecyloxy)propan-1-ol (0.67 g, 0.0022 mol) was then added along with 3 mL of DMF and this slurry was concentrated to remove water. The slurry was then treated with N,N′-Diisopropylcarbodiimide (0.748 mL, 0.00477 mol) and heated to 65° C. for 4 h. The crude mixture was concentrated and chromatographed on silica with 30% MeOH/methylene chloride, 0.3% ammonia. The partially purified material was rechromatographed with 20% MeOH: 80% methylene chloride containing 0.4% aqueous ammonia. Fractions that were clean by HPLC were combined and concentrated and dried over high vacuum overnight. The material was transferred to a vial and blown down which results in the formation of a solid. This was pumped down under high vacuum for 4 days to provide 36 in 20% yield from 35. MS (ESI+) for C₃₀H₅₅N₆O₄P m/z 595.2 (M+H)⁺.

### Example 33

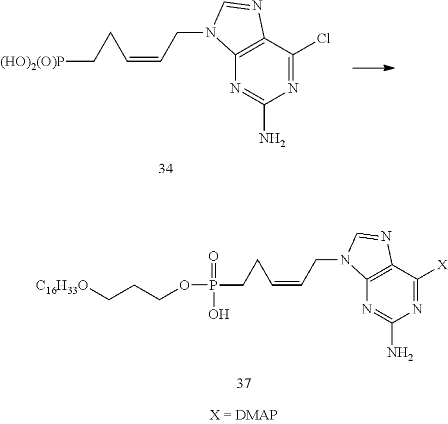

[(3Z)-5-(2-amino-6-chloro-9H-purin-9-yl)pent-3-en-1-yl]phosphonic acid 34 (0.513 g, 0.00161 mol) and 3-(hexadecyloxy)propan-1-ol (0.686 g, 0.00228 mol) were dissolved in N,N-dimethylformamide (4.5 mL, 0.059 mol) and partially concentrated to make sure there is no water present. Enough DMF was then back added to make up the volume. 4-Dimethylaminopyridine (0.30 g, 0.0024 mol) was added followed by N,N′-Diisopropylcarbodiimide (0.741 mL, 0.00473 mol). The mixture was stirred at 50° C. for 1 hour and then heated to 70° C. and stirred overnight. The reaction mixture was concentrated and chromatographed on silica with 25% MeOH/CH₂Cl₂, 2% NH₄OH to afford 37 as a yellow solid foam in 33% yield. MS (ESI+) for C₃₆H₆₀N₇O₄P m/z 686.5 (M+H)⁺.

### Example 34

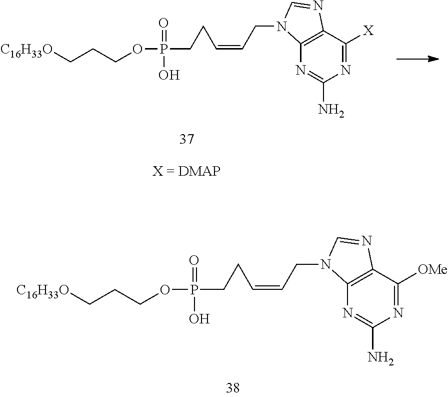

DMAP adduct of purine 37 (0.420 g, 0.000518 mol) in dimethyl sulfoxide (10 mL, 0.1 mol) and methanol (0.1000 mL, 0.002469 mol) was treated with sodium hydride (79 mg, 0.0020 mol) at room temperature. The mixture was stirred for 10 minutes and the solution became a slurry. This was heated to 70° C. overnight and there was barely any product formed. The reaction mixture was cooled to room temperature and treated with 10 mL of MeOH and lithium hydroxide (0.035 g, 0.0015 mol) and stirred at room temperature for 15 min. The mixture was concentrated and chromatographed on silica gel with CH₂Cl₂: MeOH:ammonia 80:20:0.5 to afford the desired product. This material was taken up in methylene chloride and filtered through a 2μ syringe filter. about 10 mL of MeOH was added to the solution along with a few drops of formic acid and the solution concentrated to convert the product 38 to the acid form as an amorphous solid in 58% yield. MS (ESI+) for C₃₀H₅₄N₅O₅P m/z 596.6 (M+H)⁺.

### Example 35

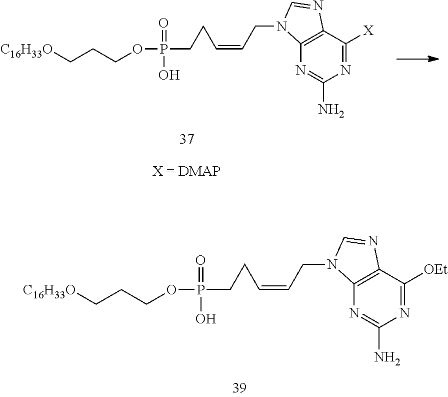

Compound 37 (0.215 g, 0.000313 mol) in ethanol (5.0 mL, 0.086 mol) was treated with lithium hydroxide (22 mg, 0.00094 mol) and heated to 80° C. for a few minutes to get the LiOH to dissolve. After 3 hours the solution was concentrated and chromatographed on silica with 700:150:20 CH₂Cl₂:MeOH:NH₄OH. Fractions were pooled and concentrated to afford the product 39 in 89% yield. MS (ESI+) for C₃₁H₅₆N₅O₅P m/z 610.3 (M+H)⁺.

### Example 36

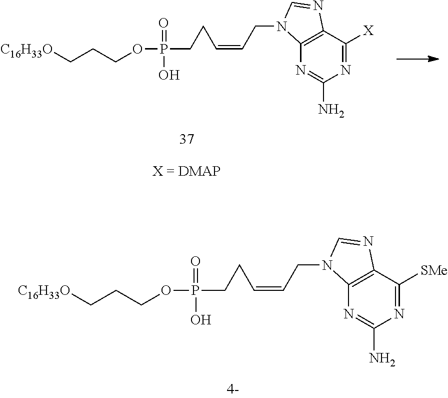

Compound 37 (0.253 g, 0.000369 mol) in N,N-dimethylformamide (2.0 mL, 0.026 mol) was treated with sodium methyl mercaptide (0.100 g, 0.00143 mol) and allowed to stir at room temperature overnight. The mixture was concentrated and chromatographed on silica with 15% MeOH—CH₂Cl₂ containing 0.4% NH₄OH. The crude material was taken up in MeOH and formic acid and concentrated to remove the ammonia. HPLC showed a more polar impurity. As a result the material was chromatographed again using the same solvent system and again treated with formic acid and concentrated to provide the product 40 in 50% yield. MS (ESI+) for C₃₀H₅₄N₅O₄PS m/z 612.3 (M+H)⁺. NMR (400 MHz, CHLOROFORM-d) ppm 0.88 (t, J=6.74 Hz, 5H) 1.12-1.39 (m, 47H) 1.43-1.60 (m, 4H) 1.69-1.93 (m, 7H) 2.42-2.67 (m, 8H) 3.39 (dt, J=47.32, 6.50 Hz, 7H) 3.84-4.15 (m, 3H) 4.50-4.82 (m, 3H) 5.30-5.88 (m, 3H) 7.75 (s, 2H) 8.12 (s, 3H);

Since modifications will be apparent to those of skill in the art, it is intended that the invention is limited only by the scope of the appended claims.

1. A compound of Formula (I) or Formula (II): 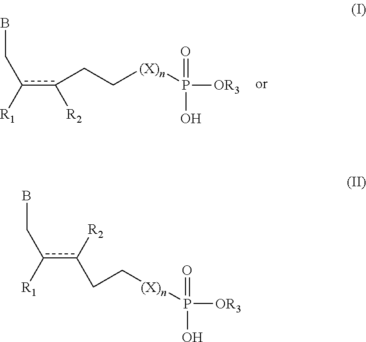
or salts, solvates or hydrates thereof wherein:
B is a purine or pyrimidine base or an analog thereof;
R₁ and R₂ are independently hydrogen, alkyl, substituted alkyl, aryl, substituted aryl, arylalkyl, substituted arylalkyl, R₄CO₂—, R₅O—, R₆S(O)ₖ—, halo, heteroalkyl, —N₃, substituted heteroalkyl, heteroaryl, substituted heteroaryl, heteroarylalkyl or substituted heteroarylalkyl or alternatively, in the compound of Formula (I), R₁ and R₂ together with the carbon atoms to which they are bonded form a cycloalkyl, substituted cycloalkyl, cycloheteroalkyl or substituted cycloheteroalkyl ring;
X is —CH₂—;
n is 0 or 1;
k is 0, 1 or 2;
R₃ is hydrogen, a monovalent cation or a lipophilic group; and
R₄, R₅, and R₆ are independently alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl;
provided that at least one of R₁ and R₂ is not hydrogen or that when both R₁ and R₂ are hydrogen, B is 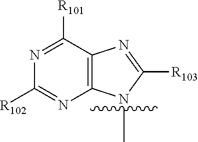
wherein R₁₀₁ is —OR₁₀₄, —SR₁₀₅, —NR₁₀₆NH₂ or —NR₁₀₇NHSO₂Me, R₁₀₂ is hydrogen, alkyl, halo or —NR₁₀₈R₁₀₉, R₁₀₃ is hydrogen or alkyl and R₁₀₄, R₁₀₅, R₁₀₆, R₁₀₇, R₁₀₈ and R₁₀₉ are independently hydrogen or alkyl;
provided that when R₁ and R₂ together with the atoms to which they are bonded form a cycloalkyl, substituted cycloalkyl, cycloheteroalkyl or substituted cycloheteroalkyl ring a double bond is optionally present between the carbon atoms connecting R₁ and R₂; and
provided that when R₁ and R₂ are independently hydrogen, alkyl, substituted alkyl, aryl, substituted aryl, arylalkyl, substituted arylalkyl, R₄CO₂—, R₅O—, R₆S(O)ₖ—, halo, heteroalkyl, —N₃, substituted heteroalkyl, heteroaryl, substituted heteroaryl, heteroarylalkyl or substituted heteroarylalkyl a double bond is present between the carbon atoms connecting R₁ and R₂

2. The compound of claim 1, wherein R₃ is n-C₁₄H₂₉O(CH₂)₃—, n-C₁₅H₃₁O(CH₂)₃—, n-C₁₆H₃₃O(CH₂)₃—, n-C₁₇H₃₅O(CH₂)₃— or n-C₁₈H₃₇O(CH₂)₃—.

3. The compound of claim 1, wherein B is

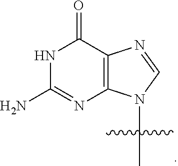 [SMILES: *n1cnc2c(=O)[nH]c(N)nc21]

4. The compound of claim 1, wherein R₂ is alkyl or halo.

5. The compound of claim 1, wherein R₂ is methyl or fluoro.

6. The compound of claim 1, wherein R₁ and R₂ together with the atoms to which they are bonded form a cycloalkyl or substituted cycloalkyl ring.

7. The compound of claim 1, wherein R₁ and R₂ together with the atoms to which they are bonded form a cyclopropyl or substituted cyclopropyl ring.

8. The compound of claim 1, wherein R₁ and R₂ together with the atoms to which they are bonded form

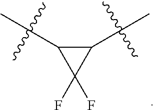 [SMILES: *C1C(*)C1(F)F]

9. The compound of claim 1, wherein R₃ is n-C₁₆H₃₃—O—(CH₂)₃— and B is

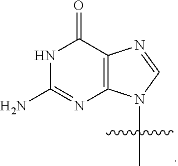 [SMILES: *n1cnc2c(=O)[nH]c(N)nc21]

10. The compound of claim 1, wherein R₃ is n-C₁₆H₃₃O(CH₂)₃— and R₂ is alkyl or halo.

11. The compound of claim 1, wherein R₃ is n-C₁₆H₃₃O(CH₂)₃— and R₂ is methyl or fluoro.

12. The compound of claim 1, wherein R₃ is n-C₁₆H₃₃O(CH₂)₃— and R₁ and R₂ together with the atoms to which they are bonded form a cycloalkyl or substituted cycloalkyl ring.

13. The compound of claim 1, wherein R₃ is n-C₁₆H₃₃O(CH₂)₃— and R₁ and R₂ together with the atoms to which they are bonded form

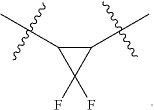 [SMILES: *C1C(*)C1(F)F]

14. The compound of claim 1, wherein B is 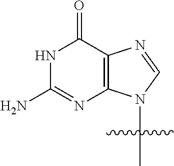 [SMILES: *n1cnc2c(=O)[nH]c(N)nc21]
and R₂ is alkyl or halo.

15. The compound of claim 1, wherein B is 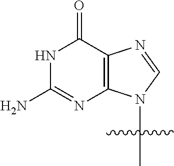 [SMILES: *n1cnc2c(=O)[nH]c(N)nc21]
and R₂ is methyl or fluoro.

16. The compound of claim 1, wherein B is 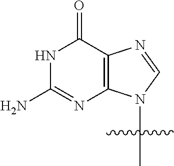 [SMILES: *n1cnc2c(=O)[nH]c(N)nc21]
and R₁ and R₂ together with the atoms to which they are bonded form a cycloalkyl or substituted cycloalkyl ring.

17. The compound of claim 1, wherein B is 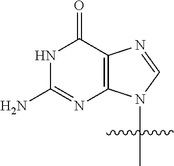 [SMILES: *n1cnc2c(=O)[nH]c(N)nc21]
and R₁ and R₂ together with the atoms to which they are bonded form

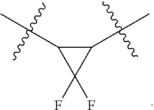 [SMILES: *C1C(*)C1(F)F]

18. The compound of claim 1, wherein R₃ is n-C₁₆H₃₃O(CH₂)₃—, B is 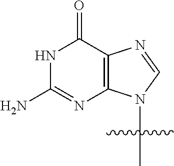 [SMILES: *n1cnc2c(=O)[nH]c(N)nc21]
and R₂ is alkyl or halo.

19. The compound of claim 1, wherein R₃ is n-C₁₆H₃₃O(CH₂)₃—, B is 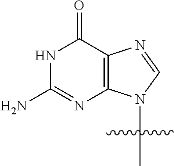 [SMILES: *n1cnc2c(=O)[nH]c(N)nc21]
and R₂ is methyl or fluoro.

20. The compound of claim 1, wherein R₃ is n-C₁₆H₃₃O(CH₂)₃⁻, B is 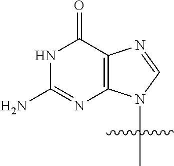 [SMILES: *n1cnc2c(=O)[nH]c(N)nc21]
and R₁ and R₂ together with the atoms to which they are bonded form a cycloalkyl or substituted cycloalkyl ring.

21. The compound of claim 1, wherein R₃ is n-C₁₆H₃₃O(CH₂)₃⁻, B is 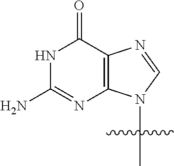 [SMILES: *n1cnc2c(=O)[nH]c(N)nc21]
and R₁ and R₂ together with the atoms to which they are bonded form

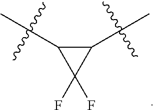 [SMILES: *C1C(*)C1(F)F]

22. The compound of claim 1, wherein R₃ is n-C₁₆H₃₃O(CH₂)₃— and B is 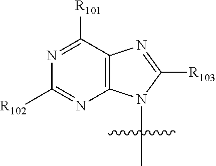
wherein R₁₀₁ is −OR₁₀₄, —SR₁₀₅, —NR₁₀₆NH₂ or —NR₁₀₇NHSO₂Me, R₁₀₂ is hydrogen, alkyl, halo or —NR₁₀₈R₁₀₉, R₁₀₃ is hydrogen or alkyl and R₁₀₄, R₁₀₅, R₁₀₆, R₁₀₇, R₁₀₈ and R₁₀₉ are independently hydrogen or alkyl and R₁ and R₂ are hydrogen.

23. The compound of claim 21, wherein R₁₀₄, R₁₀₅, R₁₀₆, R₁₀₇ are alkyl and R₁₀₂ is —NR₁₀₈R₁₀₉.

24. The compound of claim 21, wherein R₁₀₄, R₁₀₅, R₁₀₆, R₁₀₇ are alkyl and
R₁₀₂ is —NR₁₀₈R₁₀₉ and R₁₀₃ is hydrogen.

25. The compound of claim 21, wherein R₁₀₄, R₁₀₅, R₁₀₆, R₁₀₇ are methyl or ethyl and R₁₀₂ is —NH₂ and R₁₀₃ is hydrogen.

26. The compound of claim 21, wherein R₁₀₄ is methyl or ethyl, R₁₀₅, R₁₀₆, R₁₀₇ are methyl, R₁₀₂ is —NH₂ and R₁₀₃ is hydrogen.

27. (canceled)

28. (canceled)

29. (canceled)

30. (canceled)

31. A pharmaceutical composition comprising the compound of claim 1 and a pharmaceutically acceptable vehicle.

32. A method of preventing or treating a viral infection comprising administering to a subject in need thereof a therapeutically effective amount of the compound of claim 1.

33. (canceled)

34. (canceled)

35. (canceled)
